(19) United States
(12) Patent Application Publication
(10) Pub. No.: US 2026/0097150 A1
(43) Pub. Date: Apr. 9, 2026

(54) Implantable Device for Treating an Ocular Condition

(71) Applicant: Celanese EVA Performance Polymers LLC, Irving, TX (US)

(72) Inventors: Harsh Patel, Union, KY (US); Jeffrey Haley, Norwood, OH (US); Cyonna Holmes, Irving, TX (US)

(21) Appl. No.: 19/348,856

(22) Filed: Oct. 3, 2025

Related U.S. Application Data
(60) Provisional application No. 63/704,047, filed on Oct. 7, 2024.

(51) Int. Cl. A61L 27/54; A61L 27/18

(52) U.S. Cl. CPC A61L 27/54; A61L 27/18; A61L 2300/222

(57) ABSTRACT

The implantable device includes a core including a core polymer matrix within which is dispersed one or more therapeutic agents. The core polymer matrix includes a hydrophobic polymer comprising a first poly ortho ester (POE) polymer. The POE polymer has a Tg of between about −20° C. and 40° C. as determined in accordance with ASTM E1640-18. Methods for treating conditions in a patient in need thereof are also provided.

### RELATED APPLICATION

The present application is based upon and claims priority to U.S. Provisional Patent Application Ser. No. 63/704,047, having a filing date of Oct. 7, 2024, which is incorporated herein by reference.

### BACKGROUND OF THE INVENTION

Drug delivery to the eye, particularly delivery of sufficient amounts of drugs to affected eye tissues can be difficult. For example, while eye drops are useful for treating diseases that occur on the outer surface of the eye or tissue(s) in the anterior chamber of the eye, eye drops cannot penetrate significantly into the eye, which is required for the treatment of various retinal and choroidal diseases. Direct injection into the eye using conventional needles and syringes can be effective, but often require frequent injections over time in order to administer therapeutically effective amounts of a drug to the tissue.

As such, a need currently exists for better options and techniques to deliver therapeutically effective amounts of drugs to ocular tissue over a sustained period of time.

### SUMMARY OF THE INVENTION

In accordance with one embodiment of the present invention, an implantable device for treatment of an ocular condition is disclosed. The implantable device includes a core including a core polymer matrix within which one or more therapeutic agents are dispersed. The core polymer matrix includes a hydrophobic polymer including a first poly ortho ester (POE) polymer. The POE polymer has a T_{g} of between about −20° C. and 40° C. as determined in accordance with ASTM E1640-18.

Other features and aspects of the present invention are set forth in greater detail below.

### BRIEF DESCRIPTION OF THE DRAWINGS

A full and enabling disclosure of the present invention, including the best mode thereof, directed to one of ordinary skill in the art, is set forth more particularly in the remainder of the specification, which makes reference to the appended drawings in which:

FIG. 1 is a perspective view of one embodiment of the implantable medical device of the present disclosure;

FIG. 2 is a cross-sectional view of the implantable medical device of FIG. 1;

FIG. 3 is a perspective view of one embodiment of the implantable medical device of the present disclosure;

FIG. 4 is a cross-sectional view of the implantable medical device of FIG. 3;

FIG. 5 is a perspective view of one embodiment of the implantable medical device of the present disclosure;

FIG. 6 is a perspective view of another embodiment of the implantable medical device of the present disclosure;

FIG. 7 is a cross-sectional view of the implantable medical device of FIG. 6;

FIG. 8 is an exploded view of one embodiment of the implantable device of the present disclosure;

FIG. 9 is an exploded view of one embodiment of the implantable device of the present disclosure;

FIG. 10 is an exploded view of one embodiment of the implantable device of the present disclosure;

FIG. 11 is a perspective view of an implantable medical device, specifically a punctal plug, according to an embodiment of the present disclosure; and

FIG. 12 is a graph depicting the cumulative release of IgG from an example embodiment of an implantable medical device;

FIG. 13 is a graph depicting the cumulative release of trastuzumab from Example 2;

FIG. 14 is a graph depicting the cumulative release of trastuzumab from Example 2;

FIG. 15 is an HPLC chromatogram taken before extrusion for Example 2;

FIG. 16 is an HPLC chromatogram taken after extrusion for Example 2;

FIG. 17 are light microscopy images taken for Example 3;

FIG. 18 are light microscopy images taken for Example 4;

FIG. 19 is a graph depicting the cumulative release of IgG from Examples 3-4;

FIG. 20 is a graph depicting the cumulative release of IgG from Examples 3-4;

FIG. 21 are light microscopy images taken for Example 5;

FIG. 22 are light microscopy images taken for Example 6;

FIG. 23 is a graph depicting the cumulative release of axinitib from Examples 5-6;

FIG. 24 is a graph depicting the cumulative release of axinitib from Examples 5-6;

FIG. 25 is a DSC of the first POE polymer of Example 7;

FIG. 26 is a DSC of the second POE polymer of Example 7; and

FIG. 27 is a DSC of the POE polymer blend of Example 7.

Repeat use of references characters in the present specification and drawing is intended to represent same or analogous features or elements of the invention.

### DETAILED DESCRIPTION

It is to be understood by one of ordinary skill in the art that the present discussion is a description of exemplary embodiments only and is not intended as limiting the broader aspects of the present invention.

Generally speaking, the present disclosure is directed to an implantable device that is capable of delivering one or more therapeutic agents that can prohibit and/or treat a condition, disease, and/or cosmetic state in a patient in need thereof (e.g., human, pet, farm animal, etc.). The implantable device contains a polymer matrix (e.g., core polymer matrix) that includes a hydrophobic polymer and a pharmaceutical formulation that is dispersed within the polymer matrix. The hydrophobic polymer can include a POE polymer. The POE polymer can have a glass transition temperature ranging from about −20° C. to 40° C. as determined in accordance with ASTM E1640-18. The POE polymer can have a molecular weight ranging from about 1 kDa to about 100 kDa.

Through selective control over the particular nature and concentration of the components of the implantable device, the present inventors have discovered that the resulting device can be effective for sustained release of an antibody over a prolonged period of time. For example, the implantable device can release one or more therapeutic agents from about 20 days to about 210 days, and in some embodiments, from about 30 days to about 180 days.

Various embodiments of the present invention will now be described in more detail.

### I. Core

### A. Polymer Matrix

As indicated above, the implantable device includes a core formed from a polymer matrix. The polymer matrix includes at least one hydrophobic polymer that can retain its structural integrity for a certain period of time when placed in an aqueous environment, such as the body of a mammal, and stable enough to be stored for an extended period before use. The polymer matrix can be utilized to form a core or core polymer matrix for an implantable device as is discussed further hereinbelow. The hydrophobic polymer can include silicone polymer, polyolefins, polyvinyl chloride, polycarbonates, polysulphones, styrene acrylonitrile copolymers, polyurethanes, silicone polyether-urethanes, polycarbonate-urethanes, silicone polycarbonate-urethanes, ethylene vinyl acetate copolymers, etc., as well as combinations thereof. The hydrophobic polymer can comprise from about 30 wt. % to about 80 wt. %, such as from about 40 wt. % to about 70 wt. %, such as from about 30 wt. % to about 60 wt. % of the core.

The hydrophobic polymer can include a biodegradable polymer. Examples of suitable biodegradable polymers for this purpose may include, for instance, polymers made of monomers such as organic esters or ethers, which are capable of degradation in a variety of environments. Anhydrides, amides, ortho esters, by themselves or in combinations with other monomers can also be used. The polymers can be crosslinked or non-crosslinked. The biodegradable polymers can also include various groups such as oxygen-based groups, including oxy, hydroxy, ether, carbonyl, e.g., non-oxo-carbonyl, ester, carboxylic acids, etc., amide cyano, amino, etc. Suitable biodegradable polymers useful for drug delivery are described in Heller, Biodegradable Polymers in Controlled Drug Delivery, In: “CRC Critical Reviews in Therapeutic Drug Carrier Systems”, Vol. 1. CRC Press, Boca Raton, Fla. (1987), which is incorporated by reference herein in its entirety. Specifically, suitable biodegradable polymers include polylactic acid polymers (PLA), poly(D,L) lactide polymers, poly ortho ester (POE) polymers, etc. as well as combinations thereof.

In certain embodiments, the polymer matrix includes a POE polymer. POE polymers contain ortho ester groups, which are functional groups containing three alkoxy groups attached to one carbon atom, along the backbone of the polymer. POE polymers can undergo surface erosion via a hydrolysis mechanism. Suitable POE polymers for use in the present disclosure are further described in U.S. Pat. Nos. 4,093,709A, 4,180,646, and 4,304,767, which are incorporated herein by reference. The POE polymers of the present disclosure can have glass transitions temperatures (T_{g}) ranging from about −20° C. to about 40° C., such as from about −20° C. to about 35° C., such as from about −10° C. to about 30° C., such as from about 0° C. to about 25° C., such as from about 5° C. to about 20° C. as determined in accordance with ASTM E1640-18. The POE polymers of the present disclosure can have a weight-average molecular weight ranging from about 1 kDa to 100 kDa, such as from about 5 kDa to about 80 kDa, such as from about 20 kDa to about 50 kDa, such as from about 20 kDa to about 70 kDa.

POE polymers can be synthesized via a variety of processes, including, condensation reactions of furans and dialcohols. POE polymers can also be polymerized via the transesterification of other esters with polyols. Additionally, POE polymers can be polymerized via addition reactions of acetals with various alcohols and/or polyols and, optionally, cyclic esters (e.g., glycolide and/or lactide). In embodiments herein, the POE polymers can include the reaction product formed from an addition reaction with one or more acetal monomers, one or more polyol monomers, and, optionally, one or more cyclic esters. The cyclic esters can be conjugated to the one or more polyol monomers, forming a modified polyol monomer. Such modified polyol monomers can add ester functionality to the POE polymer. As further described below, controlling the nature and amounts of acetals, polyols (including modified polyols), and additional monomers (e.g., cyclic ester monomers), provides for selective control over the total quantity of each monomer in the POE polymer and the total quantity of ester functional groups in the POE polymer. Suitable addition reactions of ketene acetals and polyols are described in U.S. Pat. Nos. 4,304,767, 4,549,010, and 5,968,543, which are incorporated herein by reference. For instance, the POE polymer is produced by copolymerizing an acetal monomer and a polyol monomer in a suitable solvent (e.g., polar solvents). The selected monomers can be dispersed in a polar solvent with trace amounts of acidic catalyst added as needed. The degree of polymerization during the addition reaction can be controlled via adjusting the stoichiometry of the monomers according to the Carothers Equation, shown below.

$$DP = \frac{1 + r}{1 + r - 2 rp}$$

where DP is the average degree of polymerization, r is the excess of one monomer to the other and p is the degree of conversion of the polymerization. Further, in order to control the molecular weight of the POE polymer, one or more monofunctional monomers (e.g., an alcohol instead of a diol) can be added at the beginning of the polymerization. Reaction of the monofunctional alcohol with the ketene acetal monomer blocks the polymerization of the ketene acetal monomer at one end.

In embodiments, the POE polymer can be polymerized by placing one or more acetal monomers and one or more polyol monomers in a suitable solvent in a paddle-stirred flask under anhydrous conditions. Optionally, the anhydrous conditions can be maintained while a catalyst is added. The mixture is stirred for about one hour. The POE polymer can then be isolated from the solution by either precipitation into n-hexane followed by filtration of the white solid or by evaporation of the tetrahydrofuran in Teflon coated pan placed in a vacuum chamber. Suitable solvents include aprotic solvents, such as dimethylacetamide, dimethyl sulfoxide, dimethylformamide, acetonitrile, acetone, ethyl acetate, pyrrolidone, tetrahydrofuran, and methylbutyl ether, and the like. Catalysts are not required for this reaction, but when used, suitable catalysts are iodine in pyridine, p-toluenesulfonic acid; salicylic acid, Lewis acids (such as boron trichloride, boron trifluoride, boron trichloride etherate, boron trifluoride etherate, stannic oxychloride, phosphorous oxychloride, zinc chloride, phosphorus pentachloride, antimony pentafluoride, stannous octoate, stannic chloride, diethyl zinc, and mixtures thereof); and Brønsted catalysts (such as polyphosphoric acid, crosslinked polystyrene sulfonic acid, acidic silica gel, and mixtures thereof). In embodiments, the amount of catalyst used can be about 0.2% by weight relative to the acetal monomer. Smaller or larger amounts can also be used, such as about 0.005% to about 2.0% by weight relative to the acetal monomer.

As noted, acetal monomers can be used to polymerize the POE polymers. As used herein “acetal” refers to compounds including both acetals and ketals. Suitable acetal monomers can include aliphatic acetals, aryl acetals, cyclic acetals, cyclic ketene acetals (CKAs), including bicyclic ketene acetals, and combinations thereof. Suitable ketene acetals are shown below.

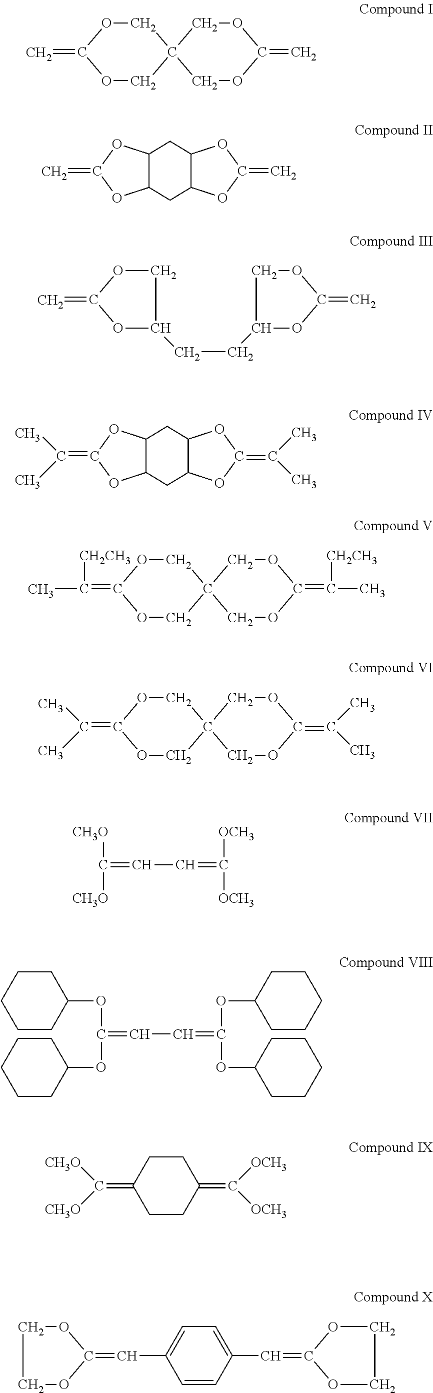

In embodiments, the acetal monomer includes a bicyclic ketene acetal monomer. A suitable bicyclic ketene acetal monomer is 3,9-diethylidene-2,4,8,10-tetraoxaspiro[5.5]undecane (DETOSU), as shown below in Formula 1.

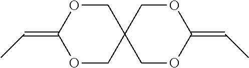

DETOSU has a chemical formula of C₁₁H₁₆O₄ and is derived from the isomeric allyl acetal 3,9-divinyl-2,4,8,10-tetraoxaspior[5.5]undecane (DVTOSU). DETOSU is a bifunctional monomer and is capable of forming POE polymers by the addition of polyols (e.g., diols) to the activated double bond of the diketene acetal. The acetals monomers used herein can include a variety of functionalities including those having a functionality of at least two or greater. It is to be understood that where the acetal monomer has functionalities greater than two, crosslinking of the polymer will likely result. The acetal monomers used herein can include a single acetal monomer or blends and/or combinations of different acetal monomers. The acetal monomer content of the POE polymer may be within a range of from about 20 mol. % to about 90 mol. %, such as from about 30 mol. % to about 80 mol. %, such as from about 40 mol. % to about 60 mol. %. In a specific embodiment, the acetal monomer content of the POE polymer is about 45 mol. % to about 55 mol %, such as about 50 mol. %.

POE polymers of the present disclosure can be synthesized using polyol monomers. As used herein, “polyol” refers to a compound having more than one hydroxyl group. Suitable polyols include triols and the like that can enter into the polymerization reaction without adversely affecting it or the polymeric product. Generally, suitable polyols can include a, w-aliphatic diols, triols and the like of the straight or branched chain type. Representative polyols are alkane polyols having a terminal hydroxyl group at the terminus of an alkylene chain of the formula:

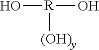

wherein R is an alkylene chain of 2 to 12 carbon atoms and y is 0 to 6. Typical diols include 1,5-pentanediol; 1,6-hexanediol; 1,7-heptanediol; 1,9-nonanediol; 2,3-dimethyl-1,6-hexanediol; 3,6-diethyl-1,9-nonanediol; 1,12-dodecamethanediol; and the like.

Polyols containing more than 2 reactive hydroxyl radicals suitable for use herein include polyhydroxyl compounds such as 1,2,3,4,5,6-hexanehexol; 1,2,3-propanetriol; 1,2,5-pentanetriol; 1,3,5-pentanetriol; 1,2,4-butanetriol; 2-methyl-1,2,3-propanetriol; 2-methyl-2 (hydroxymethyl) 1,2-propanediol; 1,4,7-heptanetriol; 1,5,10-decanetriol; 1,5,12-dodecanetriol; and the like.

Other polyols suitable for synthesizing the polymers include polyglycols containing a repeating glycol monoether moiety —OCH2 (CH₂)ₚOH wherein p is 1 to 5, and the polyglycols are diglycols, triglycols, tetraglycols, and the like. Typical polyglycols include diethylene glycol, triethylene glycol, tetraethylene glycol, bis(4-hydroxybutyl) ether, bis(3-hydroxypropyl) ether, and the like.

Additional polyols that can be used in accordance with the disclosure are polyhydroxyl compounds having 2 or more reactive hydroxyl groups such as pentaerythritol; dipentaerythritol; β-methylglycerol; 1,4-cyclohexane dicarbinol in the cis, trans isomeric configuration or mixtures thereof; 2,2,4,4-tetramethyl cyclobutane 1,3-diol; adonitol; mannitol; 2,5-dipropyl-1,4-phenyldipropanol; 1,3-cyclopropanol; 2-propenyl-1,4-cyclohexane dipropanol; trimethylol propane; sorbitol; penacol; 2-methyl-1,4-cyclohexane dicarbinol; 3-isopropoxy-1,4-cyclohexane dipropanol; 2-ethenyl-1,3-cyclopentane dicarbinol; 1,4-phenyldicarbinol; 2-propyl-1,4-phenyldiethanol; 3-butoxy-1,4-phenyldibutanol; and the like. The preparation of the above polyols is known to the art and described further in Acta Pharm. Jugaslav. Vol 2 pages 134 to 139, 1952; Ann. Vol. 594, pages 76 to 88, 1955; J. Am. Chem. Soc. Vol 71, pages 3618 to 3621, 1949; ibid., Vol. 74, pages 2674 to 2675, 1952; Chem. Abst., Vol. 42, pages 8774 to 8775, 1948; ibid., Vol 43 pages 571 to 573 and 6652, 1949; ibid., Vol. 44, pages 2554 and 7231, 1950; ibid., Vol. 46, page 9585, 1952; ibid., Vol. 47, page 7575, 1953; ibid., Vol. 48, page 106, 1954, ibid., Vol. 49, pages 6098 to 6099, 1955; Encyclopedia of Chemical Technology, Kirk-Othmer, Vol. 10, pages 638 to 678, 1966, published by Interscience Publishers, New York.

Phenolic polyols (two or more phenolic hydroxyl groups) and mixed phenolic-alcoholic polyols may be employed. Also, mixtures of two or more polyols may be employed. Examples of polyols and of mixed phenolic-alcoholic polyols are as follows: 4,4′-isopropylidenediphenol (bisphenol A); 4-hydroxybenzylalcohol; 4-hydroxy-3-methoxybenzylalcohol; p-hydroxyphenethylalcohol; 4,4′-dihydroxydiphenyl; 4,4′-dihydroxydiphenylmethane; 2,4-dihydroxybenzaldehyde; catechol; resorcinol; hydroquinone; 2,2′-dihydroxybenzophenone; 2,4-dihydroxybenzophenone; and 3,4-dihydroxymethylcinnamate; also non-phenolic polyols having aromatic linking groups between the hydroxyl groups, e.g. 1,4-dihydroxymethylbenzene. Furthermore, tri-(and higher) hydric phenols may be used such as pyrogallol; hydroxyhydroquinone; phloruglucinol; and propyl gallate.

In embodiments, the polyols include hexane-1,6-diol (HDO), cyclohexane dimethanol (CHDM), triethylene glycol (TEG), and combinations thereof. Similar to the acetal monomers, the polyol monomers can have functionalities of at least 2 or greater. It is to be understood that where the polyol monomer has functionalities greater than two, crosslinking of the polymer will likely result. The polyol monomers used herein can include a single polyol monomer or blends and/or combinations of different polyol monomers. The polyol monomer content of the POE polymer may be within a range of from about 20 mol. % to about 90 mol. %, such as from about 30 mol. % to about 80 mol. %, such as from about 40 mol. % to about 60 mol. %. In a specific embodiment, the polyol monomer content of the POE polymer is about 45 mol. % to about 55 mol %, such as about 50 mol. %.

The polyol monomer(s) can be modified to include a polyol having one or more cyclic esters (e.g., glycolide) conjugated thereto. For instance, modified polyols can include prepolymers formed from a reaction between one or more cyclic esters and the polyol (e.g., diol). Thus, the resulting modified polyols can have aliphatic ester units and ester functionality. These modified polyols containing one or more cyclic esters can then be polymerized with one or more acetals to form the POE polymer. For instance, in embodiments, HDO, CHGM, and/or TEG can be reacted with glycolide (GL) forming the following modified polyol monomers CHDM-GL, HDO-GL, and TEG-GL. Resulting modified polyols monomers can then be reacted with acetal monomers to form the POE polymers. Other modified polyols can also be utilized in accordance with the present disclosure. For instance, the polyol monomers can be modified to include different functionalities in order to modify end properties of the POE polymer. In embodiments, from about 0 mol. % to about 100 mol. % of the polyol monomers used to form the POE polymer can include one or more modified polyols, such as from about 0 mol. % to about 75 mol. %, such as from about 10 mol. % to about 60 mol. %, such as from about 20 mol. % to about 50 mol. %. In certain embodiments, the amount of modified polyol can vary based on the type of modified polyol utilized. For instance, in embodiments where CHDM-GL is utilized, the amount of CHDM-GL is greater than about 10 mol. % of the total polyol monomers, such as between about 10 mol. % and about 50 mol. %, such as from about 15 mol. % and about 45 mol. %, such as from about 20 mol. % to about 40 mol. %. In other embodiments, where HDO-GL is utilized, the amount of HDO-GL is less than about 10 mol. % of the total polyol monomers. Indeed, as provided herein, the amount of modified polyol included can vary based on the type of modified polyol selected.

As discovered by the present inventors and further described herein, through control of the type and amount of monomers utilized during polymerization, properties of the POE polymer can be tailored to achieve desired therapeutic agent release from the polymer matrix. For instance, POE polymers can include blocks of acidic monomers (e.g., cyclic esters such as glycolide and/or lactide) distributed throughout the POE polymer backbone. These acidic segments can release acidic byproducts upon hydrolysis of the polymer, which can further catalyze hydrolysis of the POE polymer. Selection of the particular nature and amount of polyol can be used to adjust the glass transition temperature T_{g} and/or the hydrophilicity of the resultant POE polymer. For instance, in order to increase the glass transition T_{g} temperature of the POE polymer higher amounts of CHDM or modified-CHDM (e.g., CHDM-GL) can be utilized. To increase the hydrophilicity of the POE polymer, TEG or modified TEG (e.g., TEG-GL) including higher amounts of TEG can be utilized during polymerization. Further, the type and/or amount of ester functional groups throughout the POE polymer backbone can be modified to tune therapeutic agent release. To increase the ester content of the POE polymer, higher amounts of GL-modified polyols can be incorporated. For instance, HDO-GL can be utilized to increase the overall ester content of the POE polymer. Thus, selection of the particular monomers and amounts of the monomers can be used to adjust mechanical and thermal properties of the POE polymer.

In certain embodiments, it may also be desirable to employ blends of POE polymers having different mechanical and thermal properties. For instance, the polymer matrix can include a first POE polymer and a second POE polymer such that the overall blend and polymer matrix have a T_{g}, and/or molecular weight within the range noted above. For example, the polymer matrix may contain a first biodegradable polymer (e.g., POE polymer) and a second biodegradable polymer (e.g., POE polymer) having a T_{g} that is greater than the T_{g} of the first polymer. The second polymer may likewise have a molecular weight that is the same, lower, or higher than the corresponding molecular weight of the first polymer. For instance, the first POE polymer can have a T_{g} that is from about −10° C. to about 50° C., such as from about 0° C. to about 30° C., such as from about 10° C. to about 30° C. The first POE polymer can have a T_{g} that is greater than 0° C. The second POE polymer can have a T_{g} ranging from 0° C. to about 50° C., such as from about 10° C. to about 40° C., such as from about 15° C. to about 35° C. In such embodiments, the second biodegradable polymer can also include a molecular weight that is greater than the molecular weight of the first biodegradable polymer. The first polymer may constitute from about 20 wt. % to about 80 wt. %, in some embodiments from about 30 wt. % to about 70 wt. %, and in some embodiments, from about 40 wt. % to about 60 wt. % of the polymer matrix, and the second polymer may likewise constitute from about 20 wt. % to about 80 wt. %, in some embodiments from about 30 wt. % to about 70 wt. %, and in some embodiments, from about 40 wt. % to about 60 wt. % of the polymer matrix.

Further, in embodiments, the amount of ester functionality in the polymer matrix can be controlled by blending a first POE polymer having a high ester content with a second POE polymer having a lower ester content as compared to the first POE polymer. Control of the amount of ester functionality in the resultant POE polymer mixture can affect the hydrolyzation and degradation of the POE polymer. For instance, ester linkages in the backbone of the POE polymer are more easily hydrolyzed as compared to other functional groups. Hydrolysis of the ester group produces acid that catalyzes further decomposition of the POE polymer. Thus, POE polymers having greater amounts of ester in the polymer or polymer blend will degrade faster that those containing lesser amounts of esters.

Typically, biodegradable polymer(s), such as POE polymers, constitute the entire polymer matrix (e.g., core polymer matrix). In other words, the polymer matrix may be substantially free of non-biodegradable, hydrophobic polymers, such as silicone polymer, polyolefins, polyvinyl chloride, polycarbonates, polysulphones, styrene acrylonitrile copolymers, polyurethanes, silicone polyether-urethanes, polycarbonate-urethanes, silicone polycarbonate-urethanes, ethylene vinyl acetate copolymers, etc., as well as combinations thereof. If used, additional hydrophobic polymers (e.g., those used in addition to POE polymers) generally constitute no more than 10 wt. % of the polymer matrix, in some embodiments no more than about 5 wt. % of the polymer matrix, and in some embodiments, from 0 wt. % to about 2 wt. % of the polymer matrix (e.g., 0 wt. %).

In certain embodiments, the polymer matrix can further include one or more hydrophilic polymers, such as celluloses, including ethylcellulose, methylcellulose, hydroxymethylcellulose, etc., polyvinylpyrrolidone, and so forth. In such embodiments, the POE polymers may be blended with other types of hydrophilic polymers. In such embodiments, POE polymers may constitute from about from about 70 wt. % to about 99.999 wt. %, in some embodiments from about 80 wt. % to about 99.99 wt. %, and in some embodiments, from about 90 wt. % to about 99.9 wt. % of the polymer content of the polymer matrix, while other hydrophilic polymers constitute from about 0.001 wt. % to about 30 wt. %, in some embodiments from about 0.01 wt. % to about 20 wt. %, and in some embodiments, from about 0.1 wt. % to about 10 wt. % of the polymer content of the polymer matrix.

### B. Additives

The core may also optionally contain one or more additives or excipients if so desired, such as radiocontrast agents, release modifiers, bulking agents, plasticizers, surfactants, crosslinking agents, flow aids, colorizing agents (e.g., chlorophyll, methylene blue, etc.), antioxidants, stabilizers, lubricants, other types of antimicrobial agents, preservatives, etc. to enhance properties and processability. When employed, the optional excipient(s) typically constitute from about 0.01 wt. % to about 20 wt. %, and in some embodiments, from about 0.05 wt. % to about 15 wt. %, and in some embodiments, from about 0.1 wt. % to about 10 wt. % of the core. In one embodiment, for instance, a radiocontrast agent may be employed to help ensure that the device can be detected in an X-ray based imaging technique (e.g., computed tomography, projectional radiography, fluoroscopy, etc.). Examples of such agents include, for instance, barium-based compounds, iodine-based compounds, zirconium-based compounds (e.g., zirconium dioxide), etc. One particular example of such an agent is barium sulfate. Other known antimicrobial agents and/or preservatives may also be employed to help prevent surface growth and attachment of bacteria, such as metal compounds (e.g., silver, copper, or zinc), metal salts, quaternary ammonium compounds, etc. The core can also be formulated to have a desired flexural modulus of elasticity ranging from about 2 MPa to about 200 MPa.

To help further control the release rate from the implantable medical device, a hydrophilic compound may also be incorporated into the core that is soluble and/or swellable in water. When employed, the weight ratio of the POE polymer(s) the hydrophilic compounds within the core may range about 0.25 to about 200, in some embodiments from about 0.4 to about 80, in some embodiments from about 0.8 to about 20, in some embodiments from about 1 to about 16, and in some embodiments, from about 1.2 to about 10. Such hydrophilic compounds may, for example, constitute from about 1 wt. % to about 65 wt. %, in some embodiments from about 2 wt. % to about 50 wt. %, and in some embodiments, from about 5 wt. % to about 40 wt. % of the core, while POE polymer(s) typically constitute from about 40 wt. % to about 99 wt. %, in some embodiments from about 50 wt. % to about 98 wt. %, and in some embodiments, from about 60 wt. % to about 95 wt. % of the core.

Suitable hydrophilic compounds may include, for instance, polymers, non-polymeric materials (e.g., glycerin, saccharides, sugar alcohols, salts, etc.), etc. Examples of suitable hydrophilic polymers include, for instance, sodium, potassium and calcium alginates, carboxymethylcellulose, agar, gelatin, polyvinyl alcohols, polyalkylene glycols (e.g., polyethylene glycol), collagen, pectin, chitin, chitosan, poly-1-caprolactone, polyvinylpyrrolidone, poly(vinylpyrrolidone-co-vinyl acetate), polysaccharides, hydrophilic polyurethane, polyhydroxyacrylate, dextran, xanthan, hydroxypropyl cellulose, methylcellulose, proteins, ethylene vinyl alcohol copolymers, water-soluble polysilanes and silicones, water-soluble polyurethanes, etc., as well as combinations thereof. Particularly suitable hydrophilic polymers are polyalkylene glycols, such as those having a molecular weight of from about 100 to 500,000 grams per mole, in some embodiments from about 500 to 200,000 grams per mole, and in some embodiments, from about 1,000 to about 100,000 grams per mole. Specific examples of such polyalkylene glycols include, for instance, polyethylene glycols, polypropylene glycols polytetramethylene glycols, polyepichlorohydrins, etc. Examples of sugar alcohols include sugar alcohols (e.g., xylitol, sorbitol, mannitol, maltitol, erythritol, galactitol, isomalt, inositol, lactitol, etc.). In embodiments, the core includes one or more sugar alcohols (e.g., mannitol).

### C. Therapeutic Agent(s)

One or more therapeutic agents are also dispersed within the polymer matrix that are capable of prohibiting and/or treating a condition, disease, and/or cosmetic state of a patient. Therapeutic agents are distinguished from other components such as matrices, carriers, diluents, excipients, additives, etc. or other protective components. The therapeutic agent may be prophylactically, therapeutically, and/or cosmetically active, systemically or locally. The therapeutic agent can be homogenously dispersed within the polymer matrix.

Typically, therapeutic agents will constitute from about 20 wt. % to about 80 wt. %, in some embodiments from about 30 wt. % to about 70 wt. %, and in some embodiments, from about 20 wt. % to about 60 wt. % of the core polymer matrix, while the polymer constitutes from about 20 wt. % to about 80 wt. %, in some embodiments from about 30 wt. % to about 70 wt. %, and in some embodiments, from about 30 wt. % to about 50 wt. % of the core polymer matrix. Suitable therapeutic agents will be further discussed hereinbelow.

General examples of therapeutic agents include, but are not limited to, anti-infectives (including antibiotics, antivirals, fungicides, scabicides or pediculicides); antiseptics (e.g., benzalkonium chloride, benzethonium chloride, chlorhexidine gluconate, mafenide acetate, methylbenzethonium chloride, nitrofurazone, nitromersol and the like); steroids (e.g., estrogens, progestins, androgens, adrenocorticoids, and the like); antiallergenics (e.g., sodium chromoglycate, antazoline, methapyriline, chlorpheniramine, cetrizine, pyrilamine, prophenpyridamine); miotics and anti-cholinesterase (e.g., pilocarpine, salicylate, carbachol, acetylcholine chloride, physostigmine, eserine, diisopropyl fluorophosphate, phospholine iodine, demecarium bromide); hormonal agents (e.g., estrogens, estradiol, progestational, progesterone, insulin, calcitonin, parathyroid hormone, peptide and vasopressin hypothalamus releasing factor); analgesics and anti-inflammatory agents (e.g., aspirin, ibuprofen, naproxen, ketorolac, COX-1 inhibitors, COX-2 inhibitors, salicylate, indomethacin, diclofenac, flurbiprofen, piroxicam, other non-steroidal anti-inflammatories, and the like); narcotics (e.g., morphine, meperidine, codeine, and the like); local anesthetics (e.g., the amide- or anilide-type local anesthetics such as bupivacaine, dibucaine, mepivacaine, procaine, lidocaine, tetracaine, and the like); anti-proliferative agents (e.g., 1,3-cis retinoic acid); decongestants (e.g., phenylephrine, naphazoline, tetrahydrazoline); antineoplastics (e.g., carmustine, cisplatin, fluorouracil); immunological drugs (e.g., vaccines and immune stimulants); chemotherapeutic agents (e.g., mechlorethamine, cyclophosphamide, fluorouracil, thioguanine, carmustine, lomustine, melphalan, chlorambucil; streptozocin, methotrexate, vincristine, bleomycin, vinblastine, vindesine, dactinomycin, daunorubicin, doxorubicin, tamoxifen, and the like); immunosuppressive agents, growth hormone antagonists, growth factors (e.g., epidermal growth factor, fibroblast growth factor, platelet derived growth factor, transforming growth factor beta, somatotropin, fibronectin); inhibitors of angiogenesis (e.g., angiostatin, anecortave acetate, thrombospondin, etc.); interferons (e.g., interferon alpha-2b, peg interferon alpha-2a, interferon alpha-2b+ribavirin, pegylated interferon-2a, interferon beta-1a, interferon beta); interleukins (e.g., interleukin-2); vaccines (e.g., whole viral particles, recombinant proteins, subunit proteins, gp41, gp120, gp140, DNA vaccines, plasmids, bacterial vaccines, polysaccharides, extracellular capsular polysaccharides); dopamine agonists; radiotherapeutic agents; therapeutic polypeptides and/or proteins (e.g. insulin, erythropoietin, morphogenic proteins such as bone morphogenic protein, and the like); enzymes; extracellular matrix components; ACE inhibitors; free radical scavengers; chelators; antioxidants; anti-polymerases; photodynamic therapy agents; gene therapy agents; corticosteroids (e.g., dexamethasone); tyrosine kinase inhibitors (e.g., axitinib, bosutinib, cabozantinib, crizotinib, dasatinib, erlotinib, gefitinib, imatinib, lapatinib, nilotinib, pazopanib, ponatinib, ruxolitinib, sorafenib, sunitinib, vatalanib, vemurafenib, etc.); beta blockers (e.g., timolol); and so forth, as well as combinations of any of the foregoing.

In certain embodiments, the therapeutic agent may be a macromolecular compound having a relatively large molecular weight, such as about 1 kilodaltons (kDa) or more, in some embodiments from about 2 kDa to about 1000 kDa, in some embodiments from about 20 kDa to about 950 kDa, in some embodiments from about 50 kDa to about 750 kDa, and in some embodiments, from about 100 kDa to about 500 kDa, or any range therebetween. The macromolecular compound may, for instance, include a protein, peptide, enzyme, antibody, interferon, interleukin, blood factor, vaccine, nucleotide, lipid, or an analogue, derivative, or combination thereof. Alternatively, small molecule therapeutic agents may also be employed, such as those having a molecular weight of less than about 1,000 Da, in some embodiments about 900 Da or less, in some embodiments from about 10 to about 800 Da, and in some embodiments, from about 20 to about 700 Da, or any range therebetween.

The weight ratio of the therapeutic agent to the polymer matrix can range from about 0.3 to about 2, such as from about 0.5 to about 1.75, such as from about 0.7 to about 1.5, such as from about 1 to about 1.2. The therapeutic agent can comprise from about 20 wt. % to about 80 wt. % of the device, such as from about 30 wt. % to about 70 wt. %, such as from about 20 wt. % to about 50 wt. %.

In embodiments, the therapeutic agent includes one or more antibodies. As used herein, the term “antibody” (Ab) generally includes, by way of example, both naturally occurring and non-naturally occurring Abs, monoclonal and polyclonal Abs, chimeric and humanized Abs; human or nonhuman Abs, wholly synthetic Abs, single chain Abs, etc. A nonhuman Ab may be humanized by recombinant methods to reduce its immunogenicity in man. The term “antibody” also includes an antigen-binding fragment or an antigen-binding portion of any of the disclosed immunoglobulins or peptides, and includes a monovalent and a divalent fragment or portion, and a single chain Ab. Particularly suitable antibodies may include monoclonal antibodies (“MAbs”), multispecific (e.g., bispecific) antibodies, or combinations thereof. The term “monoclonal antibody” generally refers to a non-naturally occurring preparation of Ab molecules of single molecular composition, i.e., Ab molecules whose primary sequences are essentially identical, and which exhibits a single binding specificity and affinity for a particular epitope. Multispecific antibodies, on the other hand, can bind simultaneously to different antigens (e.g., two antigens). Such antibodies are generally produced by hybridoma, recombinant, transgenic or other techniques known to those skilled in the art. A “human” antibody refers to an Ab having variable regions in which both the framework and complementarity-determining regions (CDRs) are derived from human germline immunoglobulin sequences. Furthermore, if the Ab contains a constant region, the constant region also is derived from human germline immunoglobulin sequences. The human Abs may include amino acid residues not encoded by human germline immunoglobulin sequences (e.g., mutations introduced by random or site-specific mutagenesis in vitro or by somatic mutation in vivo). However, the term “human antibody”, as used herein, is not intended to include Abs in which CDR sequences derived from the germline of another mammalian species, such as a mouse, have been grafted onto human framework sequences.

In some embodiments, the antibody (including a fragment thereof) can neutralize, block, inhibit, abrogate, reduce, and/or interfere with one or more biological activities (e.g., mitogenic, angiogenic and/or vascular permeability) of a proliferating cell. Such antibodies may, for instance, bind to HER2, TNF-α, VEGF-A, α4-integrin, CD20, CD52, CD25, CD11a, EGFR, respiratory syncytial virus (RSV), glycoprotein IIb/IIIa, IgG1, IgE, complement component 5 (C5), B-cell activating factor (BAFF), CD19, CD30, interleukin-1 beta (IL1β), prostate specific membrane antigen (PSMA), CD38, RANKL, GD2, SLAMF7 (CD319), proprotein convertase subtilisin/kexin type 9 (PCSK9), dabigatran, cytotoxic T-lymphocyte-associated protein 4 (CTLA4), interleukin-5 (IL-5), programmed cell death protein (PD-1), VEGFR2 (KDR), protective antigen (PA) of B. anthracis, interleukin-17 (IL-17), interleukin-6 (IL-6), interleukin-6 receptor (IL6R), interleukin-12 (IL-12), interleukin 23 (IL-23), sclerostin (SOST), myostatin (GDF-8), activin receptor-like kinase 1, delta like ligand 4 (DLL4), angiopoietin 3, VEGFR1, selectin, oxidized low-density lipoprotein (oxLDL), platelet-derived growth factor receptor beta, neuropilin 1, Von Willebrand factor (vWF), neural apoptosis-regulated proteinase 1, beta-amyloid, reticulon 4 (RTN4)/Neurite Outgrowth Inhibitor (NOGO-A), nerve growth factor (NGF), LINGO-1, myelin-associated glycoprotein, etc., as well as combinations thereof.

Antibodies of the present disclosure can also include multispecific antibodies, such as bispecific antibodies (BsAbs). Multispecific antibodies refer to any antibody that can bind simultaneously to two or more different antigens, while BsAbs refers to an antibody that can bind simultaneously to two different antigens. One example BsAb is a bispecific T cell engager (BiTE) with one arm targeting CD3 on T cells and the other recognizing target proteins on tumor cells, thereby activating the T cells to kill the tumor cells. In addition to their interaction with T cells, BsABs have also been designed to engage other effector ells, such as natural killer (NK) cells and macrophages for cancer therapy. Suitable BsAbs include blinatumomab (BLINCYTO™) which targets both CD19 and CD3 and catumaxomab (Removab™) which targets human EpCAM and human CD3 receptors. Other suitable BsAbs include AMG 330 (anti-CD3/CD33), AMG 427 (anti-CD3/FLT3), AMG 673 (anti-CD3/CD33), AMG 701 (anti-CD3/BCMA), AMG 160 (anti-CD3/prostate specific membrane antigen (PSMA)), AMG 596 (anti-CD3/epidermal growth factor receptor (EGFR) and AMG 757 (anti-CD3/DLL-3), all developed by Amgen.

If desired, in some embodiments, the implantable device may be suited to deliver a nucleic acid as a therapeutic agent. The therapeutic agent can include one or more nucleic acids. As used herein, the term “nucleic acid” generally refers to a compound comprising a nucleobase and an acidic moiety, e.g., a nucleoside, nucleotide, polynucleotide, or a combination thereof. A “nucleoside” generally refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” generally refers to a nucleoside including a phosphate group. Modified nucleotides may by synthesized by any useful method, such as, for example, chemically, enzymatically, or recombinantly, to include one or more modified or non-natural nucleosides. Polynucleotides may comprise a region or regions of linked nucleosides. Such regions may have variable backbone linkages. The linkages may be standard phosphodiester linkages, in which case the polynucleotides would comprise regions of nucleotides. For example, polynucleotides may contain three or more nucleotides in which adjacent nucleotides are linked to each other via a phosphodiester linkage. The term “nucleic acid” also encompasses RNA as well as single and/or double-stranded DNA. More particularly nucleic acids may be or may include, for example, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-c-LNA having a 2′-amino functionalization), ethylene nucleic acids (ENA), cyclohexenyl nucleic acids (CeNA) or chimeras or combinations thereof.

Nucleic acids may be naturally occurring, for example, in the context of a genome, a transcript, a mRNA, tRNA, rRNA, siRNA, snRNA, plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or including non-naturally occurring nucleotides or nucleosides. Nucleic acids can be purified from natural sources, produced using recombinant expression systems and optionally purified, chemically synthesized, etc. The nucleic acids may also include nucleoside analogs, such as analogs having chemically modified bases or sugars, and backbone modifications. In some embodiments, the nucleic acid is or contains natural nucleosides (e.g., adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadeno sine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

Modified nucleotide base pairing may be employed and encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures. One example of such non-standard base pairing is the base pairing between the modified nucleotide inosine and adenine, cytosine or uracil. Any combination of base/sugar or linker may be incorporated into polynucleotides of the present disclosure.

In certain embodiments, the nucleic acid may be a polynucleotide (e.g., RNA polynucleotides, such as mRNA polynucleotides) in which one or more nucleobases has been modified for therapeutic purposes. In fact, in certain embodiments, a polynucleotide (e.g., RNA polynucleotide, such as mRNA polynucleotide) may be employed that includes a combination of at least two (e.g., 2, 3, 4 or more) modified nucleobases. For example, suitable modified nucleobases in the polynucleotide may be a modified cytosine, such as 5-methylcytosine, 5-methyl-cytidine (m5C), N4-acetyl-cytidine (ac4C), 5-halo-cytidine (e.g., 5-iodo-cytidine), 5-hydroxymethyl-cytidine (hm5C), 1-methyl-pseudoisocytidine, 2-thio-cytidine (s2C), 2-thio-5-methyl-cytidine, etc.; modified uridine, such as 5-cyano uridine, 4′-thio uridine, pseudouridine (ψ), N1-methylpseudouridine (m1ψ), N1-ethylpseudouridine, 2-thiouridine (s2U), 4′-thiouridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine (mo5U), 5-methoxyuridine, 2′-O-methyl uridine, etc.; modified guanosine, such as α-thio-guanosine, inosine (I), 1-methyl-inosine (m1I), wyosine (imG), methylwyosine (mimG), 7-deaza-guanosine, 7-cyano-7-deaza-guanosine (preQO), 7-aminomethyl-7-deaza-guanosine (preQ1), 7-methyl-guanosine (m7G), 1-methyl-guanosine (m1G), 8-oxo-guanosine, 7-methyl-8-oxo-guanosine, etc.; modified adenine, such as α-thio-adenosine, 7-deaza-adenine, 1-methyl-adenosine (m1A), 2-methyl-adenine (m2A), N6-methyl-adenosine (m6A), 2,6-diaminopurine, etc.; as well as combinations thereof. In some embodiments, the polynucleotide (e.g., RNA polynucleotide, such as mRNA polynucleotide) includes a combination of at least two (e.g., 2, 3, 4 or more) of the aforementioned modified nucleobases.

In some embodiments, the polynucleotide (e.g., RNA polynucleotide, such as mRNA polynucleotide) may be uniformly modified (e.g., fully modified, modified throughout the entire sequence) for a particular modification. For example, a polynucleotide can be uniformly modified with 5-methyl-cytidine (m5C), meaning that all cytosine residues in the mRNA sequence are replaced with 5-methyl-cytidine (m5C). Similarly, a polynucleotide can be uniformly modified for any type of nucleoside residue present in the sequence by replacement with a modified residue such as any of those set forth above.

In some embodiments, polynucleotides function as messenger RNA (mRNA). “Messenger RNA” (mRNA) generally refers to any polynucleotide that encodes a (at least one) polypeptide (a naturally occurring, non-naturally occurring, or modified polymer of amino acids) and can be translated to produce the encoded polypeptide in vitro, in vivo, in situ or ex vivo. The basic components of a mRNA molecule typically include at least one coding region, a 5′ untranslated region (UTR), a 3′ UTR, a 5′ cap and a poly-A tail. Polynucleotides may function as mRNA but can be distinguished from wild-type mRNA in their functional and/or structural design features that serve to overcome existing problems of effective polypeptide expression using nucleic-acid based therapeutics. The mRNA may contain at least one (one or more) ribonucleic acid (RNA) polynucleotide having an open reading frame encoding at least one polypeptide of interest. In some embodiments, a RNA polynucleotide of a mRNA encodes 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9 or 9-10 polypeptides. In some embodiments, a RNA polynucleotide of a mRNA encodes at least 10, 20, 30, 40, 50, 60, 70, 80, 90 or 100 polypeptides. In some embodiments, an RNA polynucleotide of a mRNA encodes at least 100 or at least 200 polypeptides.

In some embodiments, the nucleic acids are therapeutic mRNAs. As used herein, the term “therapeutic mRNA” refers to a mRNA that encodes a therapeutic protein. Therapeutic proteins mediate a variety of effects in a host cell or a subject in order to treat a disease or ameliorate the signs and symptoms of a disease. For example, a therapeutic protein can replace a protein that is deficient or abnormal, augment the function of an endogenous protein, provide a novel function to a cell (e.g., inhibit or activate an endogenous cellular activity, or act as a delivery agent for another therapeutic compound (e.g., an antibody-drug conjugate). Therapeutic mRNA may be useful for the treatment of various diseases and conditions, such as bacterial infections, viral infections, parasitic infections, cell proliferation disorders, genetic disorders, and autoimmune disorders. The mRNA may be designed to encode polypeptides of interest selected from any of several target categories including, but not limited to, biologics, antibodies, vaccines, therapeutic proteins or peptides, cell penetrating peptides, secreted proteins, plasma membrane proteins, cytoplasmic or cytoskeletal proteins, intracellular membrane bound proteins, nuclear proteins, proteins associated with human disease, targeting moieties or those proteins encoded by the human genome for which no therapeutic indication has been identified but which nonetheless have utility in areas of research and discovery.

Particularly suitable therapeutic mRNAs are those that include at least one ribonucleic acid (RNA) polynucleotide having an open reading frame encoding at least one antigenic polypeptide, in which the RNA polynucleotide of the RNA includes at least one chemical modification. The chemical modification may, for instance, be pseudouridine, N1-methylpseudouridine, N1-ethylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine,), 5-methoxyuridine, and 2′-O-methyl uridine.

Although by no means required, the particular nature of the nucleic acid may also be selected to help improve its ability to be dispersed within the polymer matrix and delivered to a patient without significant degradation. For instance, it may be desired to co-deliver a conventional RNA (e.g., mRNA) with a self-amplifying RNA. Conventional mRNAs, for instance, generally include an open reading frame for the target antigen, flanked by untranslated regions and with a terminal poly(A) tail. After transfection, they drive transient antigen expression. Self-amplifying mRNAs, on the other hand, are capable of directing their self-replication, through synthesis of the RNA-dependent RNA polymerase complex, generating multiple copies of the antigen-encoding mRNA, and express high levels of the heterologous gene when they are introduced into the cytoplasm of host cells. Circular RNA (circRNA), which is a single-stranded RNA joined head to tail, may also be employed. The target RNA may be circularized, for example, by backsplicing of a non-mammalian exogenous intron or splint ligation of 5′ and 3′ ends of a linear RNA. Examples of suitable circRNAs are described, for instance, in U.S. Patent Publication No. 2019/0345503, which is incorporated herein by reference thereto. Antisense RNA may also be employed, which generally has a base carried on a backbone subunit composed of morpholino backbone groups and in which the backbone groups are linked by inter-subunit linkages (both charged and uncharged) that allow the bases in the compound to hybridize to a target sequence in an RNA by Watson-Crick base pairing, thereby forming an RNA:oligonucleotide heteroduplex within the target sequence. Morpholino oligonucleotides with uncharged backbone linkages, including antisense oligonucleotides, are detailed, for example, in U.S. Pat. Nos. 5,698,685, 5,217,866, 5,142,047, 5,034,506, 5,166,315, 5,185,444, 5,521,063, and 5,506,337, which are incorporated herein by reference. Other exemplary antisense oligonucleotides are described in U.S. Pat. Nos. 9,464,292, 10,131,910, 10,144,762, and 10,913,947, which are incorporated herein by reference.

In certain cases, the nucleic acid may be an aptamer, such as an RNA aptamer. An RNA aptamer may be any suitable RNA molecule that can be used on its own as a stand-alone molecule, or may be integrated as part of a larger RNA molecule having multiple functions, such as an RNA interference molecule. For example, an RNA aptamer may be located in an exposed region of a shRNA molecule (e.g., the loop region of the shRNA molecule) to allow the shRNA or miRNA molecule to bind a surface receptor on the target cell. After it is internalized, it may then be processed by the RNA interference pathways of the target cell. The nucleic acid that forms the nucleic acid aptamer may include naturally occurring nucleosides, modified nucleosides, naturally occurring nucleosides with hydrocarbon linkers (e.g., an alkylene), and/or or a polyether linker (e.g., a PEG linker) inserted between one or more nucleosides, modified nucleosides with hydrocarbon or PEG linkers inserted between one or more nucleosides, or a combination of thereof. In some embodiments, nucleotides or modified nucleotides of the nucleic acid aptamer can be replaced with a hydrocarbon linker or a polyether linker. Suitable aptamers are described, for instance, in U.S. Pat. No. 9,464,293, which is incorporated herein by reference thereto.

Protein-fused nucleic acids may also be suitable for use in the present invention. For example, proteins (e.g., antibodies) may be covalently linked to RNA (e.g., mRNA). Such RNA-protein fusions may be synthesized by in vitro or in situ translation of mRNA pools containing a peptide acceptor attached to their 3′ ends. In one embodiment, after readthrough of the open reading frame of the message, the ribosome pauses when it reaches the designed pause site, and the acceptor moiety occupies the ribosomal A site and accepts the nascent peptide chain from the peptidyl-tRNA in the P site to generate the RNA-protein fusion. The covalent link between the protein and the RNA (in the form of an amide bond between 3′ end of the mRNA and the C-terminus of the protein that it encodes) allows the genetic information in the protein to be recovered and amplified (e.g., by PCR) following selection by reverse transcription of the RNA. Once the fusion is generated, selection or enrichment is carried out based on the properties of the mRNA-protein fusion, or, alternatively, reverse transcription may be carried out using the mRNA template while it is attached to the protein to avoid the impact of the single-stranded RNA on the selection. Examples of such protein-fused nucleic acids are described, for instance, in U.S. Pat. No. 6,518,018, which is incorporated herein by reference. Ribozymes (e.g., DNAzyme and/or RNAzyme) may also be employed that are conjugated to nucleic acids having a sequence that catalytically cleaves RNA, such as described in U.S. Pat. No. 10,155,946, which is incorporated herein by reference.

Apart from single strand nucleic acids such as described above, various specific types of double strand nucleic acids may also be employed to help improve stability. Circular DNA (cDNA) and plasmid nucleic acids (e.g., pDNA), which are a closed circular form of DNA, may be employed in certain embodiments. Examples of such nucleic acids are described, for instance, in WO 2004/060277 which is incorporated herein by reference. Long double stranded DNA may also be employed. For instance, a scaffolded DNA origami may be employed in which the long single-stranded DNA is folded into a certain shape by annealing the scaffold in the presence of shorter oligonucleotides (“staples”) containing segments or regions of complementary sequences to the scaffold. Examples of such structures are described, for instance, in U.S. Patent Publication Nos. 2019/0142882 and 2018/0171386, which are incorporated herein by reference.

Antisense RNA (e.g., ASOs) may also be employed, which generally has a base carried on a backbone subunit, containing one or more backbone groups (e.g., phosphorothioates or morpholino backbone groups) and in which the backbone groups are linked by inter-subunit linkages (both charged and uncharged) that allow the bases in the compound to hybridize to a target sequence in an RNA by Watson-Crick base pairing, thereby forming an RNA:oligonucleotide heteroduplex within the target sequence. ASOs are complementary to a section of naturally occurring RNA, such as mRNA or viral RNA, to form Watson-Crick base pairs and thus to inhibit a biological function of the RNA. As used herein “antisense oligonucleotide” refers to a nucleotide sequence which is substantially complementary to a target nucleotide sequence in a pre-mRNA molecule, hrRNA (heterogenous nuclear RNA), or mRNA molecule. The degree of complementarity (or substantial complementarity) of the antisense sequence is preferably such that a molecule comprising the antisense sequence can form a stable hybrid with the target nucleotide sequence in the RNA molecule under physiological conditions.

The length of the ASO can range from about 5 nucleotide or nucleoside subunits in length to about 50 nucleotide or nucleoside subunits in length. As will be appreciated, a subunit is a nucleobase and a sugar combination (e.g., ribose or deoxyribose) suitably bound to adjacent subunits through phosphorus or non-phosphorus linkages, as further discussed hereinbelow. The length of the ASO can range from about 10 subunits to about 45 subunits, such as from about 15 subunits to about 40 subunits, such as from about 20 subunits to about 35 subunits. In certain embodiments, the ASO has a length of from about 10 subunits to about 25 subunits. The length of the ASO can vary so long as it is capable of binding selectively to the intended location on an RNA target.

Natural, or unmodified, oligonucleotides are susceptible to nuclease degradation and poor protein binding. Accordingly, unmodified oligonucleotides have inefficient tissue uptake precluding their use as clinically effective therapeutic agents. Modifications to the oligonucleotides, however, have been shown to decrease nuclease degradation. For example, multiple types of modifications to internucleoside linkages in nucleotides or nucleosides can improve various properties of the ASO. Chemical modifications to the phosphodiester backbone can be made in order to improve pharmacokinetic properties, tolerability profile, and/or target binding affinity. For instance, certain modifications to the phosphodiester backbone that can be utilized in ASOs of the present disclosure include the following: phosphorothioate ODN (PS-ODN), R-isomer of PS-ODN, (Rp-PS-ODN), S-isomer of PS-ODN, (Sp-PS-ODN), methylphosphonate ODN (PM-ODN), phosphoramidate ODN (PN-ODN) where R═H or alkyl, phosphomopholidate, phosphopiperazidate, phosphorodiamidate morpholino (PMO), and peptide nucleic acids (PNA). ASOs of the present disclosure can also include tricyclo-DNAs (tcDNA) subunits as part of the oligomer. The ASO can include a nucleoside having one or more chemically modified internucleoside linkages. As used herein “chemically modified” when referring to internucleoside linkages includes any of the chemical modifications provided hereinabove with respect to (b)-(j). In certain embodiments, at least 5%, such as at least 10%, such as at least 15%, such as at least 20%, such as at least 25%, such as at least 30%, such as at least 35%, such as at least 40%, such as at least 45%, such as at least 50%, such as at least 55%, such as at least 60%, such as at least 65%, such as at least 70%, such as at least 75%, such as at least 80%, such as at least 85%, such as at least 90%, such as at least 95%, such as at least 100%, of the internucleoside linkages in the ASO are chemically modified. In other embodiments, chemically modified internucleoside linkages can be disposed about the 3′ end or 5′ end of the ASO. For instance, at least one and up to five of the internucleoside linkages extending from either the 3′ terminus or the 5′ terminus can include chemically modified internucleoside linkages. In certain embodiments, from about 50% to about 100% of the internucleoside linkages are phosphorothioate linkages (shown in (b), (c), and (d) above). In other embodiments, from about 50% to about 100% of the internucleoside linkages are morpholino subunits (shown in (i) above). Morpholino oligonucleotides with uncharged backbone linkages are detailed, for example, in U.S. Pat. Nos. 5,698,685, 5,217,866, 5,142,047, 5,034,506, 5,166,315, 5,185, 444, 5,521,063, and 5,506,337, which are incorporated herein by reference. Other exemplary antisense oligonucleotides are described in U.S. Pat. Nos. 9,464,292, 10,131,910, 10,144,762, and 10,913,947, which are incorporated herein by reference.

Modifications to the 2′ position of the sugar moiety of the nucleoside subunit is also contemplated. For example, the 2′-position on the sugar moiety can be substituted with a 2′-substituent group. Such 2′-substituent groups can include 2′-fluoro, 2′-alkoxy, 2′-aminoalkoxy, 2′-allyloxy, 2′-imidazole-alkoxy and 2′-poly(ethylene oxide). Alkoxy and aminoalkoxy groups generally include lower alkyl groups, particularly C1-C9 alkyl. Poly(ethylene glycols) are of the structure (O—CH2-CH2) n-O-alkyl. Other 2′-substituent groups can include 2′-O-methyl (2′-OMe), 2′-O-methoxyethyl (2′-MOE) and locked nucleic acid (LNA). The use of 2′-substituent groups can increase the binding affinity of the substituted oligonucleotides or can prevent nuclease degradation in vivo. Notably, at least 5%, such as at least 10%, such as at least 15%, such as at least 20%, such as at least 25%, such as at least 30%, such as at least 35%, such as at least 40%, such as at least 45%, such as at least 50%, such as at least 55%, such as at least 60%, such as at least 65%, such as at least 70%, such as at least 75%, such as at least 80%, such as at least 85%, such as at least 90%, such as at least 95%, such as at least 100% of nucleotides in the ASO can include modified 2′-substituent groups.

The ASOs of the present disclosure can also include modified bases in the nucleotide units that make up the oligonucleotides of the ASO. Such modified bases may include 6-azapyrimidines and N-2, N-6 and O-6 substituted purines including 2-aminopropyladenine. Other modified pyrimidine and purine base are expected to increase the binding affinity of oligonucleotides to a complementary strand of nucleic acid.

Notably, the ASOs of the present disclosure can include “first generation” ASOs, which refer to oligodeoxynucleotides having 2′-deoxy ribonucleotides and phosphorothioate internucleoside linkages. Suitable ASOs can also include ASOs that are oligonucleotides having a 2′-deoxy “gap” region flanked by “wings” having nucleotides with 2′-modified ribonucleotides, referred to as “gapmers”. Suitable second generation ASOs include an “MOE gapmer” in which the 2′-modified ribonucleotide is a 2′-O-methoxyethyl (2′-MOE or simply MOE) modification, and each of the internucleoside linkages is a phosphorothioate. Second generation ASOs may have a length of 20 nucleotides of which the 5 nucleotides at each terminus are 2′-MOE nucleotides and the center ten nucleotides are 2′-deoxyribonucleotides. Accordingly, these second generation ASOs are referred to as “5-10-5 MOE gapmers” since they have a 5-10-5 wing-gap-wing motif. Suitable 5-10-5 gapmers can have the following formula:

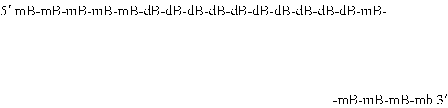

where mB=2′-O-methoxyethyl ribonucleoside; dB=deoxynucleoside; “-”=phosphorothiate linkage, and b=heterocyclic base.

The ASO can include a gap region having at least 5 to about 15, such as about 8 to about 10, contiguous 2′-deoxyribonucleosides and a first wing region and a second wing region flaking the gap region. Each of the first and second wing regions independently can include 1 to about 8 2′-O-(2-methoxyethyl) ribonucleotides. The ASOs of the present disclosure can also include “hemimers,” which are chimeric compounds in which there is a single 2′-modified “wing” adjacent to (on either 5′, or 3′ side of) a 2′-deoxy gap. The wing present in the hemimer can include 2 nucleotides to about 8 nucleotides. Notably, the gap region, first wing region, or second wing region, can include any of the internucleoside chemical modifications or 2′-substituent groups as disclosed herein.

The ASOs of the present disclosure can also include ASO conjugates, where one or more moieties are conjugated to the ASO. For instance, attached moieties can include peptides, proteins, carbohydrates, fatty acids, aptamers, and small molecules including cholesterol, tocopherol, and folic acid. Specifically, the ASO can also be conjugated to anisamide, stearic acid, cyclic RGD peptide, anandamide, N-acetylgalactosamine, spermine, and combinations thereof. For ASO conjugates, the selected moiety can be conjugated to the ASO in a variety of locations, however, in certain embodiments, the selected moiety is tethered to 3′-terminus or 5′-terminus. Different linkers can be used to conjugate the moiety to the ASO. Suitable linkers can include thiol linkers, amino linkers, aldehyde linkers, azide linkers, carboxyl linkers, dibenzylcyclooctyne linkers. ASOs (e.g., morpholino oligonucleotides) can be conjugated to arginine-rich cell penetrating peptides (CPPs). ASOs can also be conjugated to polyethylene glycol (PEG) moieties. The ASO conjugates can include saccharides, such as the monosaccharide GalNAc. For instance, up to three GalNac molecules can be attached to the ASO via a tridentate linker to 3′-terminus of the ASO. These moieties can be attached to the ASO to improve uptake mechanisms and pharmacokinetic properties of the ASO. In certain embodiments, the ASO can include a dynamic polyconjugate, which is a macromolecule having an ASO that undergoes structural modification in vivo to yield the ASO. Suitable ASO conjugates are further described in U.S. 2016/0289677, WO 2004/044141, WO 2009/073809, WO 2012/083046, WO 2012/089352, WO 2012/089602, WO 2005/086775, WO 2011/126937, WO 2009/025669, which are incorporated by reference herein.

In certain embodiments, the ASO can include an oligonucleotide that has been approved by the U.S. Food and Drug Administration (FDA). Certain approved oligonucleotides include Vitravene™ (Formiversen), Macugen™ (Pegaptanib), Kynamro™ (Mipomersen), Defitelio™ (Defibrotide), Exondys 51™ (Eteplirsen), Spinraza™ (Nusinersen), Tegsedi™ (Inotersen), Onpattro™ (Patisiran), Waylivra™ (Volanesoren), Givlaari™ (Givosiran), Vyondys 53 (Golodirsen), Viltepso™ (Vitolarsen), Oxlumo™ (Lumasiran), Leqvio™ (Inclisiran), Amondys 45™ (Casimersen), and combinations thereof. Other ASOs can include Camlingo™ (Alicaforsen), Drisapersen (PR0051, GSK-2402968), Custirsen (OGX-011 and CC-8490), Miraversen (SPC3649), ISIS-TTR02, Aezea™ (Cenersen), GTI-2040, Imetelstat (GRN163L), ISIS-STAT3RX, Liposomal Grb-2 (BP-100-1.01), and combinations thereof.

In embodiments, the nucleic acid can include an encapsulated nucleic acid that is encapsulated or coated via a carrier component. The molar ratio of the carrier component to the nucleic acid (e.g., mRNA) in the particles may vary, but is typically from about 2:1 to about 50:1, in some embodiments from about 5:1 to about 40:1, in some embodiments from about 10:1 to about 35:1, and in some embodiments, from about 15:1 to about 30:1.

As noted above, the carrier component includes a carrier, such as peptides (e.g., RALA), proteins, carbohydrates (e.g., sugars), polymers (e.g., dextran polymers, such as diethylaminoethyl-dextran; polyethyleneimine; poly(amino ester), aliphatic polyesters, such as polylactic acid, etc.), lipids, and so forth, as well as combinations of any of the foregoing, such as peptide/polymer hybrids (e.g., RALA-PLA). In one particular embodiment, for example, the carrier component may be a lipid component that includes one or more lipids. The nature of such lipid particles may generally vary as is known to those skilled in the art. In one embodiment, for example, the lipid component is a lipid vesicle (e.g., liposome) that includes one or more types and/or layers of lipids. For example, liposomes generally include a phospholipid that is capable of assembling into one or more lipid bilayers. The phospholipid has a phospholipid moiety and optionally one or more additional moieties (e.g., fatty acid moiety). The phospholipid moiety may include, for instance, phosphatidyl choline, phosphatidyl ethanolamine, phosphatidyl glycerol, phosphatidyl serine, phosphatidic acid, 2-lysophosphatidyl choline, and a sphingomyelin. When employed, the fatty acid moiety may likewise include, for instance, lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, docosahexaenoic acid, etc. Non-natural species including natural species with modifications and substitutions including branching, oxidation, cyclization, and alkynes are also contemplated. For example, a phospholipid may be functionalized with or cross-linked to one or more alkynes (e.g., an alkenyl group in which one or more double bonds is replaced with a triple bond). Under appropriate reaction conditions, an alkyne group may undergo a copper-catalyzed cycloaddition upon exposure to an azide. Such reactions may be useful in functionalizing a lipid bilayer of a nanoparticle composition to facilitate membrane permeation or cellular recognition. Examples of suitable phospholipids may include, for instance, alkyl phosphocholines, such as hexadecyl thiophosphocholine, tetradecyl phosphocholine, hexadecyl phosphocholine, docosanoyl phosphocholine, 1,2-dihexadecyl-rac-glycero-3-phosphocholine, DL-α-lysophosphatidylcholine-r-o-hexadecyl, etc.; fatty acid-modified phosphocholines, such as 1,2-distearoyl-sn-glycero-3-phosphocholine (DSPC), 1,2-dioleoyl-sn-glycero-3-phosphoethanolamine (DOPE), 1,2-dilinolenoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, etc.; as well as mixtures of any of the foregoing.

If desired, the lipid component of the vesicles may also include other types of lipids. For example, the lipid component may contain one or more structural lipids to help mitigate aggregation of other lipids in the particles. Examples of suitable structural lipids may include, for instance, steroids; sterols, such as cholesterol, fecosterol, sitosterol, ergosterol, campesterol, stigmasterol, brassicasterol, phytosterols, etc.; glycoalkaloids, such as tomatidine, tomatine, etc.; terpenoids, such as ursolic acid; tocopherols, such as alpha-tocopherol; hopanoids, stanol esters; as well as mixtures thereof. The lipid component of may also include one or more PEG-conjugated lipids to help improve the colloidal stability of the particles in biological environments by reducing a specific absorption of plasma proteins and forming a hydration layer over the particles. Examples of suitable PEG-conjugated lipids may include, for instance, PEG-modified phosphatidylethanolamines, PEG-modified phosphatidic acids, PEG-modified ceramides, PEG-modified dialkylamines, PEG-modified diacylglycerols, PEG-modified dialkylglycerols, etc., as well as mixtures thereof. For example, a PEG lipid may be (R-3-[(Ω-methoxy-poly(ethyleneglycol)2000) carbamoyl)]-1,2-dimyristyloxypropyl-3-amine) (PEG-c-DOMG), PEG-distearoyl glycerol (PEG-DMG), PEG-1,2-dipalmitoyl-sn-glycero-3-phosphocholine (PEG-DPPC), PEG-DLPE, PEG-DMPE, PEG-DSPE, etc., as well as mixtures thereof. The PEG-conjugated lipid may also be modified to include a hydroxyl group on the PEG chain to form a PEG-OH lipid. As generally defined herein, a “PEG-OH lipid” (also referred to herein as “hydroxy-PEGylated lipid”) is a PEGylated lipid having one or more hydroxyl (—OH) groups on the lipid. In certain embodiments, the PEG-OH lipid includes one or more hydroxyl groups on the PEG chain. In certain embodiments, a PEG-OH or hydroxy-PEGylated lipid includes an —OH group at the terminus of the PEG chain.

Besides lipid vesicles, other types of lipid particles may also be employed to encapsulate the nucleic acid. Solid lipid particles, for instance, may be employed in certain embodiments of the present invention. Generally speaking, such solid particles are in the form of nanoparticles having a mean diameter of from about 10 to about 1,000 nanometers, in some embodiments from about 20 to about 800 nanometers, in some embodiments from about 30 to about 600 nanometers, and in some embodiments, from about 40 to about 300 nanometers, such as determined by laser diffraction techniques. Similar to lipid vesicles, solid particles also contain a lipid component that includes one or more types and/or layers of lipids.

In one embodiment, for example, the lipid component of the solid particles includes a cationic lipid. Besides a cationic lipid, the lipid component of the solid particles may also include other types of lipids. For example, the lipid component may contain a helper lipid, which is generally neutral or non-cationic at physiological pH. Such helper lipids may include phospholipids such as described above, fatty acids, glycerolipids (e.g., mono-, di-, and triglyceride), prenol lipids, and so forth. Suitable fatty acids may include those having a fatty acid of at least 8 carbon atoms, such as unsaturated fatty acids (e.g., myristoleic acid, palmitoleic acid sapienic acid, oleic acid, elaidic acid, vaccenic acid, linoleic acid, linoelaidic acid, alpha-linoelaidic acid arachidonic acid, eicosapentaenoic acid, erucic acid, docosahexanoic acid, etc., or any cis/trans double-bond isomers thereof), saturated fatty acids (e.g., caprylic acid, capric acid, lauric acid, myristic acid, palmitic acid, stearic acid, arachidic acid, behenic acid, lignoceric acid, cerotic acid, etc., or any cis/trans double-bond isomers thereof), as well as combinations thereof. Particularly suitable helper lipids may include, for instance, oleic acid or an analog thereof, as well as fatty acid-modified phospholipids, such as 1,2-distearoyl-sn-glycero-3-phosphocholine (DSPC) and/or 1,2-dioleoyl-sn-glycero-3-phosphoethanolamine (DOPE), as well as analogs of such compounds in which the phosphocholine moiety is replaced by a different zwitterionic group, such as an amino acid or a derivative thereof.

The formation of solid lipid particles that encapsulate a nucleic acid may be accomplished by various methods as known in the art. Examples of such methods are described, for instance, in U.S. Pat. Nos. 5,795,587; 7,655,468; 7,993,672; 8,492,359; 8,771,728; and 8,956,572; as well as U.S. Patent Publication Nos. 2004/0262223; 2010/015218; 2012/0225129; 2012/0276209; 2012/0302622; 2013/0037977; 2013/0156845; 2014/0296322; and 2015/0209440, the contents of which are incorporated herein by reference thereto.

If desired, in some embodiments, the implantable device may be suited to deliver a tyrosine kinase inhibitor as a therapeutic agent. As used herein, the term “tyrosine kinase inhibitor” generally refers to a molecule, mimetic, or derivative capable of reducing, blocking, abrogating, and/or interfering with one or more biological activities, including tyrosine kinase activity. Tyrosine kinases are enzymes responsible for the activation of many proteins via signal transduction cascades. For example, tyrosine kinase inhibitors (“TKIs”) may bind to the adenosine triphosphate (ATP) binding site of VEGF resulting in blockade of intracellular signaling.

In some embodiments, the tyrosine kinase inhibitor may include, but are not limited to, an epidermal growth factor (EGF) pathway inhibitor, a platelet derived growth factor (PDGF) pathway inhibitor, a RAF-1 inhibitor, or a combination thereof. Tyrosine kinase inhibitors may include, but are not limited to, axitinib, bosutinib, cabozantinib, crizotinib, dasatinib, erlotinib, gefitinib, imatinib, lapatinib, nilotinib, pazopanib, ponatinib, ruxolitinib, sorafenib, sunitinib, vatalanib, vemurafenib, or a combination thereof. Axitinib (molecular weight of 386.5 Da), for instance, is a tyrosine kinase inhibitor that inhibits VEGF activity such as neovascularization. Thus, axitinib and other TKIs may be used for the treatment of neovascular (wet) age-related macular degeneration (AMD), the treatment of visual impairment due to diabetic macular edema (DME), the treatment of visual impairment due to macular edema secondary to retinal vein occlusion (branch RVO or central RVO), treatment of visual impairment due to choroidal neovascularization (CNV) secondary to pathologic myopia, or treatment of other retinal diseases. Cabozantinib (molecular weight of 501.5 Da), for instance, is a TKI that downregulates activation of tyrosine kinase involved in tumor angiogenesis, such as VEGF receptor.

If desired, in some embodiments, the implantable device may be suited to deliver a steroidal agent as a therapeutic agent. The steroidal agent may comprise one or more corticosteroids, such as glucocorticoids. Glucocorticoids are defined as a subgroup of corticosteroids. Glucocorticoids, sometimes also named glucocorticosteroids, are a class of steroid hormones that bind to the glucocorticoid receptor and are part of the feedback mechanism of the immune system that turns down immune activity, (e.g., inflammation). In medicine, they are used to treat diseases that are caused by an overactive immune system, such as allergies, asthma, autoimmune diseases and sepsis. They also interfere with some of the abnormal mechanisms in cancer cells, so that they are also used to treat cancer. Upon binding, the glucocorticoid receptor, the activated glucocorticoid receptor complex up-regulates the expression of anti-inflammatory proteins in the nucleus by a process known as transactivation and represses the expression of pro-inflammatory proteins in the cytosol by attenuating actions on gene induction (via NF-κB, AP1, jun-jun-homodimers, etc.).

Suitable examples of glucocorticoids may comprise hydrocortisone, cortisone acetate, cortisone/cortisol, fluorocortolone, fluocinolone, fluorometholone, prednisone, prednisolone, methylprednisolone, triamcinolone, dexamethasone, betamethasone, paramethasone, etc., as well as derivatives and combinations thereof. Dexamethasone and derivatives thereof are particularly suitable. Glucocorticoid polymorphs, isomers, hydrates, solvates, or derivatives thereof are all meant to be encompassed in the scope of the present disclosure and shall be understood to fall under the term “glucocorticoid”.

In embodiments, the therapeutic agent can include one or more prostaglandins, prostaglandin prodrug, prostaglandin analogue, and prostamide, pharmaceutically acceptable salts thereof, and mixtures thereof. Several prostaglandin analogues have been found effective as topically administered medicines in reducing the intraocular pressure, such as latanoprost, bimatoprost, tafluprost, travoprost, and unoprostone. By some experts, bimatoprost is understood as a prostaniide rather than prostaglandin derivative. Latanoprost, travoprost, tafluprost, and probably also bimatoprost, are potent and selective PGF2a agonists. Their net effect is a reduction of intraocular pressure, which is predominantly caused by a substantial increase in aqueous humor drainage, via the uveoscleral pathway. Unoprostone is sometimes also classified as a PGF2a analogue even though its potency and selectivity are much lower than in the case of the previously mentioned compounds.

In other embodiments, the therapeutic agent can include one or more Rho kinase inhibitors, Rho kinase inhibitor prodrugs, Rho kinase inhibitor analogues, pharmaceutically acceptable salts thereof, and mixtures thereof. Rho kinase (ROCK) is a kinase found in all eukaryotic cells. It regulates key processes that include cell motility, cell differentiation, cell survival, cell-cell junctions and expression of extracellular matrix proteins. Many forms of glaucoma are associated with elevated intraocular pressure (IOP) and standard treatment is to reduce IOP with drugs. Because progression of glaucoma is slow and painless, noncompliance for daily use of IOP-reducing medications is high. Rho kinase inhibitors can be utilized to suppress fibrogenic activity of trabecular meshwork to loosen the trabecular meshwork to increase aqueous humor outflow and thus, reduce IOP. Suitable Rho kinase inhibitors include ripasudil, netarsudil, and combinations thereof.

The therapeutic agent may be generally stable at high enough temperatures so that it can be incorporated into the polymer matrix at or near the melting temperature of the POE polymer(s) employed in the core without significantly degrading (e.g., melting) during manufacturing or use of the device. For example, the therapeutic agent may remain stable at temperatures of from about 20° C. to about 100° C., in some embodiments from about 25° C. to about 80° C., in some embodiments from about 30° C. to about 70° C., in some embodiments from about 35° C. to about 65° C., and in some embodiments, from about 40° C. to about 60° C. The therapeutic agent may be inherently stable at such temperatures, or it may also be encapsulated or otherwise protected by a carrier component that is stable at such temperatures, such as a carrier component containing peptides, proteins, carbohydrates (e.g., sugars), polymers, lipids, etc.

### D. Excipients

The core may also optionally contain one or more excipients, such as cell permeability enhancers, radiocontrast agents, hydrophilic compounds, bulking agents, surfactants, crosslinking agents, flow aids, colorizing agents (e.g., chlorophyll, methylene blue, etc.), antioxidants, stabilizers, lubricants, other types of antimicrobial agents, preservatives, etc. to enhance properties and processability. The optional excipient(s) typically constitute from about 0.01 wt. % to about 20 wt. %, and in some embodiments, from about 0.05 wt. % to about 15 wt. %, and in some embodiments, from about 0.1 wt. % to about 10 wt. % of the core. In one embodiment, for instance, a radiocontrast agent may be employed to help ensure that the device can be detected in an X-ray based imaging technique (e.g., computed tomography, projectional radiography, fluoroscopy, etc.). Examples of such agents include, for instance, barium-based compounds, iodine-based compounds, zirconium-based compounds (e.g., zirconium dioxide), etc. One particular example of such an agent is barium sulfate. Other known antimicrobial agents and/or preservatives may also be employed to help prevent surface growth and attachment of bacteria, such as metal compounds (e.g., silver, copper, or zinc), metal salts, quaternary ammonium compounds, etc.

To help further control the release rate from the implantable device, a hydrophilic compound may also be incorporated into the core that is soluble and/or swellable in water. When employed, the weight ratio of the POE polymer(s) within the core may range about 0.25 to about 200, in some embodiments from about 0.4 to about 80, in some embodiments from about 0.8 to about 20, in some embodiments from about 1 to about 16, and in some embodiments, from about 1.2 to about 10. Such hydrophilic compounds may, for example, constitute from about 1 wt. % to about 60 wt. %, in some embodiments from about 2 wt. % to about 50 wt. %, and in some embodiments, from about 5 wt. % to about 40 wt. % of the core, while POE polymer(s) typically constitute from about 40 wt. % to about 99 wt. %, in some embodiments from about 50 wt. % to about 98 wt. %, and in some embodiments, from about 60 wt. % to about 95 wt. % of the core. Suitable hydrophilic compounds may include, for instance, polymers, non-polymeric materials (e.g., glycerin, saccharides, sugar alcohols, salts, etc.), etc. Examples of suitable hydrophilic polymers include, for instance, sodium, potassium and calcium alginates, carboxymethylcellulose, agar, gelatin, polyvinyl alcohols, polyalkylene glycols (e.g., polyethylene glycol), collagen, pectin, chitin, chitosan, poly-1-caprolactone, polyvinylpyrrolidone, poly(vinylpyrrolidone-co-vinyl acetate), polysaccharides, hydrophilic polyurethane, polyhydroxyacrylate, dextran, xanthan, hydroxypropyl cellulose, methylcellulose, proteins, ethylene vinyl alcohol copolymers, water-soluble polysilanes and silicones, water-soluble polyurethanes, etc., as well as combinations thereof. Particularly suitable hydrophilic polymers are polyalkylene glycols, such as those having a molecular weight of from about 100 to 500,000 grams per mole, in some embodiments from about 500 to 200,000 grams per mole, and in some embodiments, from about 1,000 to about 100,000 grams per mole. Specific examples of such polyalkylene glycols include, for instance, polyethylene glycols, polypropylene glycols polytetramethylene glycols, polyepichlorohydrins, etc.

One or more nonionic, anionic, and/or amphoteric surfactants may also be employed to help create a uniform dispersion. When employed, such surfactant(s) typically constitute from about 0.05 wt. % to about 8 wt. %, and in some embodiments, from about 0.1 wt. % to about 6 wt. %, and in some embodiments, from about 0.5 wt. % to about 3 wt. % of a membrane layer. Nonionic surfactants, which typically have a hydrophobic base (e.g., long chain alkyl group or an alkylated aryl group) and a hydrophilic chain (e.g., chain containing ethoxy and/or propoxy moieties), are particularly suitable. Some suitable nonionic surfactants that may be used include, but are not limited to, ethoxylated alkylphenols, ethoxylated and propoxylated fatty alcohols, polyethylene glycol ethers of methyl glucose, polyethylene glycol ethers of sorbitol, ethylene oxide-propylene oxide block copolymers, ethoxylated esters of fatty (C₈-C₁₈) acids, condensation products of ethylene oxide with long chain amines or amides, condensation products of ethylene oxide with alcohols, fatty acid esters, monoglyceride or diglycerides of long chain alcohols, and mixtures thereof. Particularly suitable nonionic surfactants may include ethylene oxide condensates of fatty alcohols, polyoxyethylene ethers of fatty acids, polyoxyethylene sorbitan fatty acid esters, and sorbitan fatty acid esters, etc. The fatty components used to form such emulsifiers may be saturated or unsaturated, substituted or unsubstituted, and may contain from 6 to 22 carbon atoms, in some embodiments from 8 to 18 carbon atoms, and in some embodiments, from 12 to 14 carbon atoms. Sorbitan fatty acid esters (e.g., monoesters, diester, triesters, etc.) that have been modified with polyoxyethylene are one particularly useful group of nonionic surfactants. These materials are typically prepared through the addition of ethylene oxide to a 1,4-sorbitan ester. The addition of polyoxyethylene converts the lipophilic sorbitan ester surfactant to a hydrophilic surfactant that is generally soluble or dispersible in water. Such materials are commercially available under the designation TWEEN® (e.g., TWEEN® 80, or polyethylene (20) sorbitan monooleate).

In certain cases, the hydrophilic compound may be in the form of water-soluble particles distributed within the core polymer matrix. The particle size of the water-soluble particles may be controlled to help achieve the desired delivery rate. More particularly, the median diameter (D50) of the particles may be about 100 micrometers or less, in some embodiments about 80 micrometers or less, in some embodiments about 60 micrometers or less, and in some embodiments, from about 1 to about 40 micrometers, such as determined using a laser scattering particle size distribution analyzer (e.g., LA-960 from Horiba). The particles may also have a narrow size distribution such that 90% or more of the particles by volume (D90) have a diameter within the ranges noted above. In addition to controlling the particle size, the materials employed to form the water-soluble particles may also be selected to achieve the desired release profile. For example, the water-soluble particles may contain a hydroxy-functional compound that is not polymeric. The term “hydroxy-functional” generally means that the compound contains at least one hydroxyl group, and in certain cases, multiple hydroxyl groups, such as 2 or more, in some embodiments 3 or more, in some embodiments 4 to 20, and in some embodiments, from 5 to 16 hydroxyl groups. The term “non-polymeric” likewise generally means that the compound does not contain a significant number of repeating units, such as no more than 10 repeating units, in some embodiments no or more than 5 repeating units, in some embodiments no more than 3 repeating units, and in some embodiments, no more than 2 repeating units. In some cases, such a compound lacks any repeating units. Such non-polymeric compounds thus a relatively low molecular weight, such as from about 1 to about 650 grams per mole, in some embodiments from about 5 to about 600 grams per mole, in some embodiments from about 10 to about 550 grams per mole, in some embodiments from about 50 to about 500 grams per mole, in some embodiments from about 80 to about 450 grams per mole, and in some embodiments, from about 100 to about 400 grams per mole. Particularly suitable non-polymeric, hydroxy-functional compounds that may be employed in the present invention include, for instance, saccharides and derivatives thereof, such as monosaccharides (e.g., dextrose, fructose, galactose, ribose, deoxyribose, etc.); disaccharides (e.g., sucrose, lactose, maltose, etc.); sugar alcohols (e.g., xylitol, sorbitol, mannitol, maltitol, erythritol, galactitol, isomalt, inositol, lactitol, etc.); and so forth, as well as combinations thereof.

### II. Membrane Layer(s)

The implantable device can optionally include one or more membrane layers (e.g., a first membrane layer) that is positioned adjacent to an outer surface of a core. Additional membrane layers (e.g., a second membrane layer, a third membrane layer, etc.) may be layered on the core as desired. The number of membrane layers may vary depending on the particular configuration of the device, the nature of the therapeutic agent, and the desired release profile. For example, in certain embodiments, the device may contain only one membrane layer.

Regardless of the particular configuration employed, the membrane polymer matrix can include at least one hydrophobic polymer (e.g., POE polymer), such as one or more POE polymers as described hereinabove with respect to the core polymer matrix.

In certain cases, POE polymer(s) constitute the entire polymer content of the membrane polymer matrix. In other cases, however, it may be desired to include other polymers, such as other hydrophobic polymers or hydrophilic polymers. When employed, it is generally desired that such other polymers constitute from about 0.001 wt. % to about 90 wt. %, in some embodiments from about 0.01 wt. % to about 80 wt. %, and in some embodiments, from about 5 wt. % to about 75 wt. % of the polymer content of the polymer matrix. In such cases, POE polymers may constitute about from about 1 wt. % to about 99.999 wt. %, in some embodiments from about 80 wt. % to about 99.99 wt. %, and in some embodiments, from about 90 wt. % to about 99.9 wt. % of the polymer content of the polymer matrix. The membrane polymer matrix typically constitutes from about 50 wt. % to 99 wt. %, in some embodiments, from about 55 wt. % to about 98 wt. %, in some embodiments from about 60 wt. % to about 96 wt. %, and in some embodiments, from about 70 wt. % to about 95 wt. % of a membrane layer.

To help further control the release rate from the implantable medical device, a hydrophilic compound may also be incorporated into the membrane layer(s) that is soluble and/or swellable in water. When employed, the weight ratio of the POE polymer(s) the hydrophilic compounds within the membrane layer may range about 0.25 to about 200, in some embodiments from about 0.4 to about 80, in some embodiments from about 0.8 to about 20, in some embodiments from about 1 to about 16, and in some embodiments, from about 1.2 to about 10. Such hydrophilic compounds may, for example, constitute from about 1 wt. % to about 60 wt. %, in some embodiments from about 2 wt. % to about 50 wt. %, and in some embodiments, from about 5 wt. % to about 40 wt. % of the membrane layer, while POE polymer(s) typically constitute from about 40 wt. % to about 99 wt. %, in some embodiments from about 50 wt. % to about 98 wt. %, and in some embodiments, from about 60 wt. % to about 95 wt. % of the membrane layer.

Suitable hydrophilic compounds may include, for instance, polymers, non-polymeric materials (e.g., glycerin, saccharides, sugar alcohols, salts, etc.), etc. Examples of suitable hydrophilic polymers include, for instance, sodium, potassium and calcium alginates, carboxymethylcellulose, agar, gelatin, polyvinyl alcohols, polyalkylene glycols (e.g., polyethylene glycol), collagen, pectin, chitin, chitosan, poly-1-caprolactone, polyvinylpyrrolidone, poly(vinylpyrrolidone-co-vinyl acetate), polysaccharides, hydrophilic polyurethane, polyhydroxyacrylate, dextran, xanthan, hydroxypropyl cellulose, methylcellulose, proteins, ethylene vinyl alcohol copolymers, water-soluble polysilanes and silicones, water-soluble polyurethanes, etc., as well as combinations thereof. Particularly suitable hydrophilic polymers are polyalkylene glycols, such as those having a molecular weight of from about 100 to 500,000 grams per mole, in some embodiments from about 500 to 200,000 grams per mole, and in some embodiments, from about 1,000 to about 100,000 grams per mole. Specific examples of such polyalkylene glycols include, for instance, polyethylene glycols, polypropylene glycols polytetramethylene glycols, polyepichlorohydrins, etc.

Optionally, the membrane layer(s) include a plurality of water-soluble particles distributed within a membrane polymer matrix. The particle size of the water-soluble particles is controlled to help achieve the desired delivery rate. More particularly, the median diameter (D50) of the particles is about 100 micrometers or less, in some embodiments about 80 micrometers or less, in some embodiments about 60 micrometers or less, and in some embodiments, from about 1 to about 40 micrometers, such as determined using a laser scattering particle size distribution analyzer (e.g., LA-960 from Horiba). The particles may also have a narrow size distribution such that 90% or more of the particles by volume (D90) have a diameter within the ranges noted above. In addition to controlling the particle size, the materials employed to form the water-soluble particles are also selected to achieve the desired release profile. More particularly, the water-soluble particles generally contain a hydroxy-functional compound that is not polymeric. The term “hydroxy-functional” generally means that the compound contains at least one hydroxyl group, and in certain cases, multiple hydroxyl groups, such as 2 or more, in some embodiments 3 or more, in some embodiments 4 to 20, and in some embodiments, from 5 to 16 hydroxyl groups. The term “non-polymeric” likewise generally means that the compound does not contain a significant number of repeating units, such as no more than 10 repeating units, in some embodiments no or more than 5 repeating units, in some embodiments no more than 3 repeating units, and in some embodiments, no more than 2 repeating units. In some cases, such a compound lacks any repeating units. Such non-polymeric compounds thus a relatively low molecular weight, such as from about 1 to about 650 grams per mole, in some embodiments from about 5 to about 600 grams per mole, in some embodiments from about 10 to about 550 grams per mole, in some embodiments from about 50 to about 500 grams per mole, in some embodiments from about 80 to about 450 grams per mole, and in some embodiments, from about 100 to about 400 grams per mole. Particularly suitable non-polymeric, hydroxy-functional compounds that may be employed in the present disclosure include, for instance, saccharides and derivatives thereof, such as monosaccharides (e.g., dextrose, fructose, galactose, ribose, deoxyribose, etc.); disaccharides (e.g., sucrose, lactose, maltose, etc.); sugar alcohols (e.g., xylitol, sorbitol, mannitol, maltitol, erythritol, galactitol, isomalt, inositol, lactitol, etc.); and so forth, as well as combinations thereof. If utilized, the water-soluble particles typically constitute from about 1 wt. % to about 65 wt. %, in some embodiments from about 2 wt. % to about 45 wt. %, in some embodiments from about 4 wt. % to about 40 wt. %, and in some embodiments, from about 5 wt. % to about 30 wt. % of a membrane layer.

When employing multiple membrane layers, it is typically desired that each membrane layer contains a polymer matrix includes a POE polymer. Additionally, each of the membrane layers can include a plurality of water-soluble particles distributed within a membrane polymer matrix that includes a POE polymer. For example, a first membrane layer may contain first water-soluble particles distributed within a first membrane polymer matrix and a second membrane layer may contain second water-soluble particles distributed within a second membrane polymer matrix. In such embodiments, the first and second polymer matrices may each contain a POE polymer. The water-soluble particles and POE polymer(s) within one membrane layer may be the same or different than those employed in another membrane layer. In one embodiment, for instance, both the first and second membrane polymer matrices employ the same POE polymers(s) and the water-soluble particles within each layer have the same particle size and/or are formed from the same material. Likewise, the POE polymers(s) used in the membrane layer(s) may also be the same or different than the hydrophobic polymer(s) employed in the core. In one embodiment, for instance, both the core and the membrane layer(s) employ the same POE copolymer. In yet other embodiments, the membrane layer(s) may employ a POE polymer(s) having a higher T_{G} or higher molecular weight than the POE polymer employed in the core. Among other things, this can further help control the release of the therapeutic agent from the device. For instance, in embodiments the membrane layer can include a POE polymer having a T_{g} that is at least 10° C. greater, such as at least 20° C. greater, such as at least 30° C. greater, than the POE polymer(s) utilized in the core polymer matrix. In other embodiments, the membrane layer(s) may employ a POE polymer(s) having a lower T_{g} and/or molecular weight than the POE polymer(s) employed in the core.

Optionally, the membrane can include one or more porous membranes configured to facilitate release of the therapeutic agent from the device. The porous membrane can be formed from suitable materials such as polytetrafluorethylene (PTFE), polyethersulfone (PES), and combinations thereof. Suitable PFTEs include modified PTFE (mPTFE) and expanded PTFE (ePTFE). The porous membrane can include a variety of porous materials, including microporous materials. For instance, the porous membrane includes microporous ePTFE, microporous PES, and combinations thereof.

In certain other embodiments, the device can include one or more membrane layers formed form a polymer material configured to restrict release of the therapeutic agent or to direct release of the therapeutic agent to certain regions or areas of the device. For instance, certain polymeric materials can be used as membrane materials to restrict the release of the therapeutic agent. Suitable polymeric materials can include polyoxymethylene (POM), liquid crystal polymers (LCPs), and combinations thereof. Other materials that can form part or all of the membrane include metal materials, metalloids, or metal oxides, such a titanium.

If desired, membrane layer(s) used in the device may optionally contain a therapeutic agent, such as described above, which is also dispersed within the membrane polymer matrix. The therapeutic agent in the membrane layer(s) may be the same or different than the therapeutic agent employed in the core. When such a therapeutic agent is employed in a membrane layer, the membrane layer generally contains the therapeutic agent in an amount such that the ratio of the concentration (wt. %) of the therapeutic agent in the core to the concentration (wt. %) of the therapeutic agent in the membrane layer is greater than 1, in some embodiments about 1.5 or more, and in some embodiments, from about 1.8 to about 4. When employed, therapeutic agents typically constitute only from about 1 wt. % to about 40 wt. %, in some embodiments from about 5 wt. % to about 35 wt. %, and in some embodiments, from about 10 wt. % to about 30 wt. % of a membrane layer. Of course, in other embodiments, the membrane layer is generally free of therapeutic agents prior to release from the core. When multiple membrane layers are employed, each membrane layer may generally contain the therapeutic agent in an amount such that the ratio of the weight percentage of the therapeutic agent in the core to the weight percentage of the therapeutic agent in the membrane layer is greater than 1, in some embodiments about 1.5 or more, and in some embodiments, from about 1.8 to about 4.

The membrane layer(s) may also optionally contain one or more excipients as described above, such as radiocontrast agents, bulking agents, plasticizers, surfactants, crosslinking agents, flow aids, colorizing agents (e.g., chlorophyll, methylene blue, etc.), antioxidants, stabilizers, lubricants, other types of antimicrobial agents, preservatives, etc. to enhance properties and processability. When employed, the optional excipient(s) typically constitute from about 0.01 wt. % to about 60 wt. %, and in some embodiments, from about 0.05 wt. % to about 50 wt. %, and in some embodiments, from about 0.1 wt. % to about 40 wt. % of a membrane layer.

The membrane layer(s) may be formed using the same or a different technique than used to form the core, such as by hot-melt extrusion, compression molding (e.g., vacuum compression molding), injection molding, solvent casting, dip coating, spray coating, microextrusion, coacervation, etc. In other embodiments, the membrane layer(s) can be wrapped around the core and heat sealed to the core. For instance, the membrane layer(s) can be helically, radially, or longitudinally wrapped around the core and heat sealed. In one embodiment, a hot-melt extrusion technique may be employed. The core and membrane layer(s) may also be formed separately or simultaneously. In one embodiment, for instance, the core and membrane layer(s) are separately formed and then combined together using a known bonding technique, such as by stamping, hot sealing, adhesive bonding, etc. Compression molding (e.g., vacuum compression molding) may also be employed to form the implantable device. As described above, the core and membrane layer(s) may be each individually formed by heating and compressing the respective polymer compression into the desired shape while under vacuum. Once formed, the core and membrane layer(s) may be stacked together to form a multi-layer precursor and thereafter compression molded in the manner as described above to form the resulting implantable device.

### II. Implantable Device

The resulting implantable medical device may have a variety of different geometric shapes, such as cylindrical (rod), disc, ring, doughnut, helical, elliptical, triangular, ovular, etc. In one embodiment, for example, the implantable medical device may have a generally circular cross-sectional so that the overall structure is in the form of a cylinder (rod) or disc. The implantable medical device also has a relatively small size, such as a thickness (e.g., diameter) of from about 0.1 to about 10 millimeters, in some embodiments from about 0.1 to about 5 millimeters, in some embodiments from about 0.3 to about 2 millimeters, and in some embodiments, from about 0.4 to about 0.8 millimeters. The length of the implantable medical device may vary but is typically from about 1 to about 250 millimeters, in some embodiments from about 2 to about 200 millimeters, in some embodiments from about 10 to about 150 millimeters, and in some embodiments, from about 20 to about 100 millimeters.

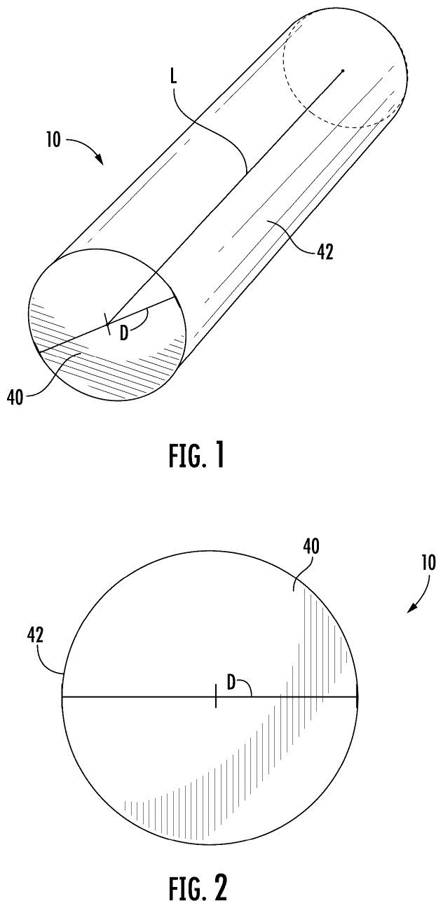

Referring to FIGS. 1-2, for example, one embodiment of an implantable device 10 is shown. The implantable device as shown in FIGS. 1-2 can be a monolithic device. As used herein, the term “monolithic” generally means that the device is formed from a single constituent layer or member, which is often referred to as a “core.” Thus, the monolithic device generally lacks additional release layers, such as shells, membranes, sheaths, etc., such that the core itself can define an exterior peripheral surface of the device. The implantable device 10 includes a core 40 having a generally circular cross-sectional shape and is elongated so that the resulting device is generally cylindrical in nature. During use of the device 10, a therapeutic agent is capable of being released from the core 40 so that it exits from the outer surface 42 of the implantable device 10.

As shown, the implantable device can have a length (L) and a cross-sectional diameter (D). The length (L) can range from about 2.5 cm to about 7 cm, such as about 3 cm to about 6 cm, such as about 4 cm to about 5 cm. In certain embodiments, the length (L) is about 5 cm. The cross-sectional diameter (D) can range from about 2 mm to about 5 mm, such as from about 3 mm to about 4 mm. In embodiments, the cross-sectional diameter is about 3.5 mm. The device can be sized according to desired therapeutic agent loading and implantation time. For example, for longer lasting implants, the size can be increased such that the implant can be loaded with enough therapeutic agent to last for the life of the implant.

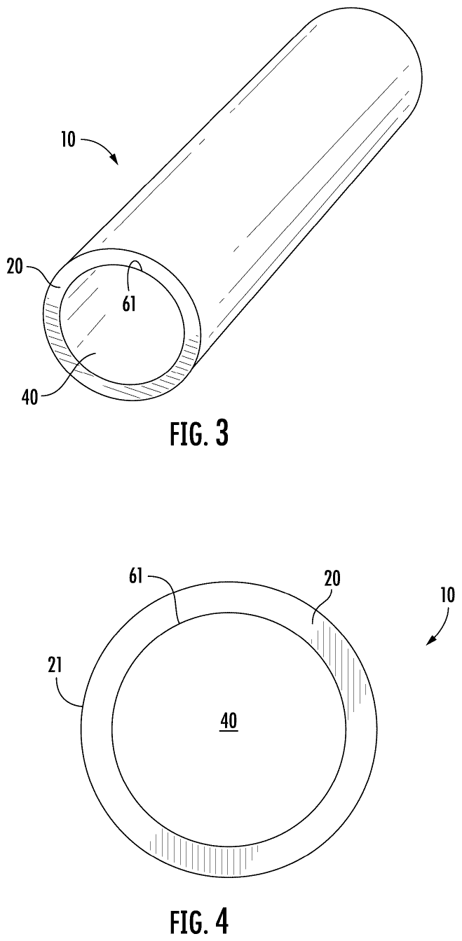

Another embodiment of an implantable device 10 is shown in FIGS. 3-4. The core 40 has a generally circular cross-sectional shape and is elongated so that the resulting device is generally cylindrical in nature. The core 40 defines an outer circumferential surface 61 about which a membrane layer 20 is circumferentially disposed. Similar to the core 40, the membrane layer 20 also has a generally circular cross-sectional shape and is elongated so that it covers the entire length of the core 40. During use of the device 10, a therapeutic agent is capable of being released from the core 40 and through the membrane layer 20 so that it exits from an external surface 21 of the device.

Of course, in other embodiments, the device may contain multiple membrane layers. In the device of FIGS. 3-4, for example, one or more additional membrane layers (not shown) may be disposed over the membrane layer 20 to help further control release of the therapeutic agent. In other embodiments, the device may be configured so that the core is positioned or sandwiched between separate membrane layers.

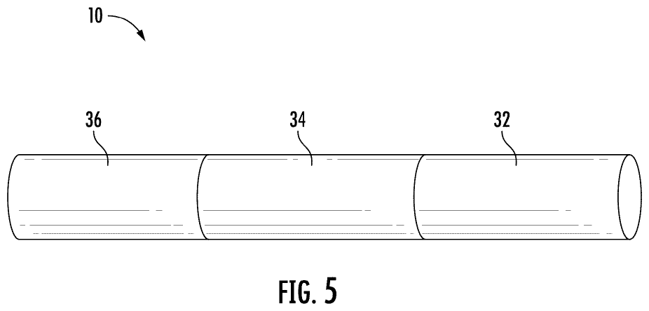

As shown in FIG. 5, the implantable device 12 can include one or more compartments. As shown, the device includes three compartments 32, 34, and 36, however, the disclosure is not so limited. Indeed, two-compartment devices are conceivable in accordance with present disclosure. In fact, any number of compartments or sections can be joined together to form an implantable device as provided herein. As shown, the implantable device 12 includes a first compartment 32, a second compartment 34, and a third compartment 36. Advantageously, the compartments 32, 34, and 36 can each be formulated to contain different amounts of therapeutic agents or different therapeutic agents depending on desired results as will be further discussed hereinbelow. It is also conceivable that the compartments 32, 24, and 36 can be formed from the same core polymer matrix or can each be formed from different core polymer matrix materials. For example, core polymer matrix materials can be modified such that the compartments can have different release rates for therapeutic agents contained therein. Furthermore, any suitable materials can be used or placed between compartments when molding the implantable device.

Multi-compartment cylinders can be formed having different types and/or amounts of therapeutic agents dispersed in each compartment. Such embodiments provide for the delivery of multiple therapeutic agents from the implantable device. In certain embodiments, the amount of therapeutic agent delivered from each compartment can vary. Indeed, each compartment may be formulated with a different core polymer matrix and/or membrane layer compositions in order to affect the release rate of therapeutic agent(s) from each compartment. For example, certain compartments can be configured to release therapeutic agent(s) faster in order to reach an initial steady state concentration, while the remaining compartments can be formulated to release therapeutic agent(s) more slowly such that sustained delivery of one or more therapeutic agent(s) over a period of time can be achieved.

Additional membrane layers can be added to the implantable device 12 of FIG. 5 as desired (not shown in FIG. 5). For example, in certain embodiments at least one membrane layer can surround the external surface of all compartments 32, 34, 36. In other embodiments, different membrane layers may surround different portions of the compartments 32, 34, and 36. For example, a first membrane can surround the first compartment 32, a second membrane can surround the second compartment 34, and a third membrane can surround the third compartment 36. The membrane layers of the compartments can be varied in order to further effect release of the dispersed therapeutic agents from the compartments.

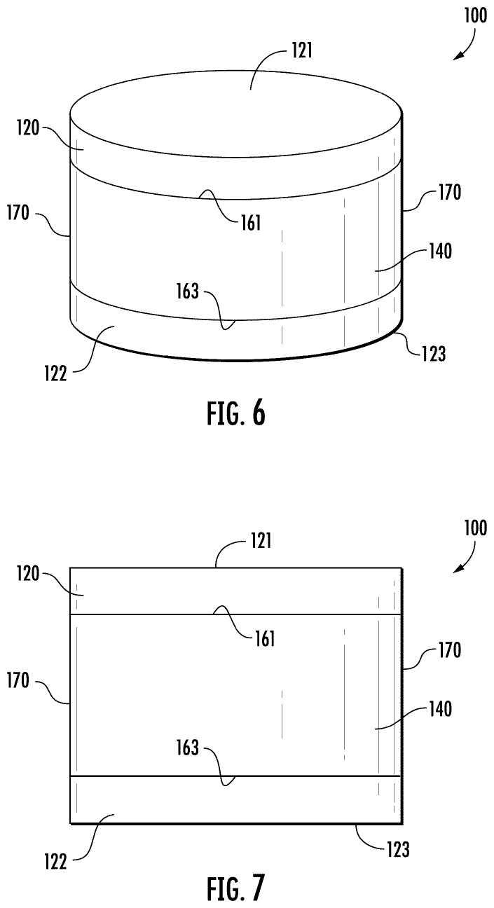

Referring now to FIGS. 6-7, for example, one embodiment of an implantable device 100 is shown that contains a core 140 having a generally circular cross-sectional shape and is elongated so that the resulting device is generally disc-shaped in nature. The core 140 defines an upper outer surface 161 on which is positioned a first membrane layer 120 and a lower outer surface 163 on which is positioned a second membrane layer 122. Similar to the core 140, the first membrane layer 120 and the second membrane layer 122 also have a generally circular cross-sectional shape that generally covers the core 140. If desired, edges of the membrane layers 120 and 122 may also extend beyond the periphery of the core 140 so that they can be sealed together to cover any exposed areas of an external circumferential surface 170 of the core 140. During use of the device 100, a therapeutic agent is capable of being released from the core 140 and through the first membrane layer 120 and second membrane layer 122 so that it exits from external surfaces 121 and 123 of the device. Of course, if desired, one or more additional membrane layers (not shown) may also be disposed over the first membrane layer 120 and/or second membrane layer 122 to help further control release of the therapeutic agent.

In other embodiments, it is contemplated that the device can be contained within a tube, the tube having one or more holes for release of the therapeutic agent. (Not shown in the Figures). The tube material can include any of the polymer materials disclosed herein or can be formed from a metallic material.

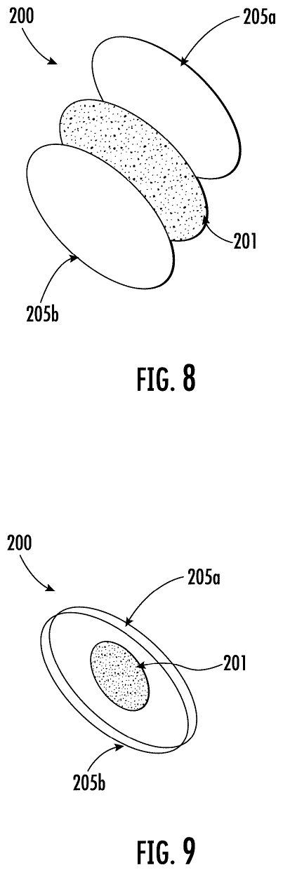

Referring to FIG. 8, one particular embodiment of an implantable device 200 is shown having a three-layer structure containing a core film layer 201 that is sandwiched between a first membrane film layer 205a and a second membrane film layer 205b. In such embodiments, the outer perimeter of the core film layer 201 is not covered by either membrane film layer 205a,205b. Accordingly, the therapeutic agent can diffuse from the core film layer 201 via the peripheral edges (e.g., circumferential edge) of the core film layer 201 and/or through membrane film layers 205a,205b. In other embodiments, however, it may be desirable to cover all surfaces, including peripheral surfaces and edges, of the core film layer with one or more membrane films. Furthermore, membrane film layer 205a and membrane film layer 205b can be of the same composition or can include different polymer matrices in order to affect drug release from the implantable device 200. FIG. 9 illustrates an implantable device 200 embodiment, where the core film layer 201 is included with (e.g., embodied within) at least one of the membrane film layers 205a. An additional membrane film layer 205b is coupled to membrane film layer 205a containing the core film layer 201. Additional membrane film layers can be utilized to cover other membrane film layers or the core film layer as desired.

Regardless of the number of layers employed, the peel strength between the core film layer and the one or more membrane film layers may be about 30 g_{f}/in or more, in some embodiments about 50 g_{f}/in or more, in some embodiments about 75 g_{f}/in or more, in some embodiments from about 80 to about 800 g_{f}/in, and in some embodiments, from about 100 to about 600 g_{f}/in, as determined using a 90° T-peel test in accordance with ASTM D1876-08 (2015). The thickness of the core and membrane film layers may also be relatively small. For example, the thickness of the core film layer may be about 0.01 mm to about 5 mm, in some embodiments from about 0.1 mm to about 4 mm, and in some embodiments from about 0.1 mm to about 2 mm. The thickness of the membrane film layer may likewise be from about 10 μm to about 500 μm, in some embodiments from about 50 μm to about 250 μm, and in some embodiments from about 100 μm to about 150 μm. The implantable medical device itself may thus be relatively small in size, such as having a total thickness (e.g., diameter) of from about 0.1 to about 10 millimeters, in some embodiments from about 0.1 to about 5 millimeters, in some embodiments from about 0.3 to about 2 millimeters, and in some embodiments, from about 0.4 to about 0.8 millimeters. The length and/or width of the implantable medical device may vary but is typically from about 1 to about 250 millimeters, in some embodiments from about 2 to about 200 millimeters, in some embodiments from about 10 to about 150 millimeters, and in some embodiments, from about 20 to about 100 millimeters.

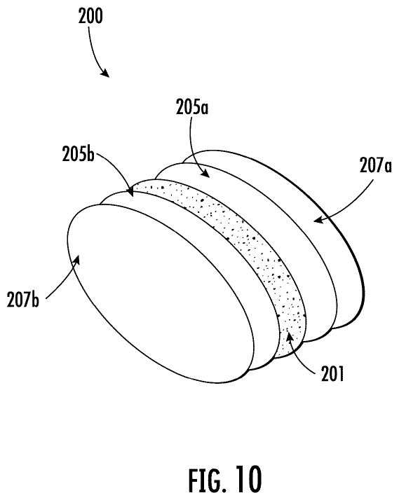

Other embodiments include a multi-layer composite having additional membrane film layers bonded to each other as shown in FIG. 10. As contemplated, FIG. 10 includes a core film layer 201 having a first membrane layer 205a and a second membrane layer 205b thereon. Additional membrane film layers 207a,207b are laminated to a surface of the first membrane layer 205a and the second membrane film layer 205b, respectively. Regardless of the number of membrane film layers employed, the peel strength between each of the membrane film layers may be about 30 g_{f}/in or more, in some embodiments about 50 g_{f}/in or more, in some embodiments about 75 g_{f}/in or more, in some embodiments from about 80 to about 800 g_{f}/in, and in some embodiments, from about 100 to about 600 g_{f}/in, as determined using a 90° T-peel test in accordance with ASTM D1876-08 (2015). While three-layered and five-layered embodiments are shown, the present disclosure is not so limited. Any number of membrane film layers can be utilized in order to produce the implantable device of the present disclosure.

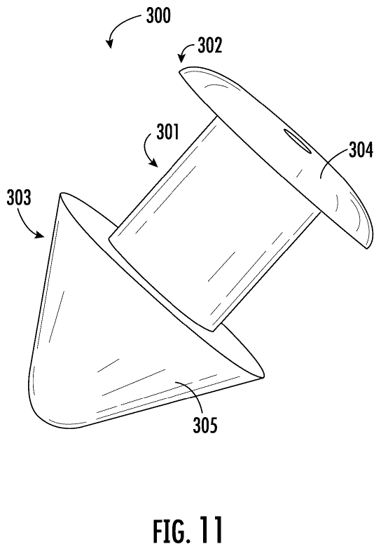

Referring to FIG. 11, in embodiments the implantable device can include a punctal plug 300. The punctal plug 300 generally includes a tubular structure 301 having a first end 302 and a second end 303. The first end 302 can include a disc 304 that is configured to secure the punctal plug 300 in a desired location within the eye. The second end 303 can include a conical member 305 that is configured to facilitate insertion of the punctal plug 300. Components of the punctal plug 300 can be formed from the polymer matrix materials disclosed herein. Further, the punctal plug 300 or portions of the punctal plug (e.g., the tubular structure 301, first end 302 and/or second end 303) can be coated with a membrane as disclosed herein above. Thus, the punctal plug 300 includes a membrane polymer matrix having a therapeutic agent dispersed therein and is capable of providing the therapeutic agent(s) in desired ocular tissue.

### III. Use of Device

Through selective control over the particular nature of the core and/or membrane layer(s), the resulting device can be effective for sustained release of the therapeutic agent over a prolonged period of time. For example, the implantable device can release the therapeutic for a time period of about 3 days or more, in some embodiments 5 days or more, in some embodiments 7 days or more, in some embodiments 10 days or more, in some embodiments about 20 days or more, in some embodiments about 30 days or more, in some embodiments about 60 days or more, in some embodiments about 90 days or more, and in some embodiments, from about 120 days to about 365 days (e.g., about 180 days). Further, the therapeutic agent can be released in a controlled manner (e.g., zero order or near zero order) over the course of the release time period. After a time period of 30 days, for example, the cumulative release ratio of the implantable device may be from about 5% to about 95%, such as from about 10% to about 90%, such as from about 15% to about 85%, such as from about 20% to about 80%, such as from about 25% to about 75%, such as from about 30% to about 70%, such as from about 35% to about 65%, such as from about 40% to about 60%, such as from about 45% to about 55%. Likewise, after a time period of 60 days, the cumulative release ratio of the implantable device may still be from about 40% to about 85%, in some embodiments from about 50% to about 80%, and in some embodiments, from about 60% to about 80%. The “cumulative release ratio” may be determined by dividing the amount of the therapeutic agent released at a particulate time interval by the total amount of therapeutic agent initially present, and then multiplying this number by 100.

Of course, the actual dosage level of the therapeutic agent delivered will vary depending on the particular therapeutic agent employed and the time period for which it is intended to be released. The dosage level is generally high enough to provide a therapeutically effective amount of the therapeutic agent to render a desired therapeutic outcome, i.e., a level or amount effective to reduce or alleviate symptoms of the condition for which it is administered. More particularly, a used herein, the phrase “therapeutically effective amount” means a dose of the therapeutic agent that results in a detectable improvement in one or more symptoms or indicia of a disease, such as retinal disease (e.g., age-related macular degeneration), or a dose of therapeutic agent that inhibits, prevents, lessens, or delays the progression of a disease. The exact amount necessary will vary, depending on the subject being treated, the age and general condition of the subject to which the therapeutic agent is to be delivered, the capacity of the subject's immune system, the degree of effect desired, the severity of the condition being treated, the particular therapeutic agent selected and mode of administration of the composition, among other factors. In one embodiment, for example, a therapeutically effective amount can be from about 0.05 mg to about 5 mg, in some embodiments from about 0.1 mg to about 4 mg, and in some embodiments, from about 0.5 to about 3 mg. The amount of the therapeutic agent contained within the individual doses may be expressed in terms of milligrams of drug per kilogram of patient body weight (i.e., mg/kg). For example, the therapeutic agent may be administered to a patient at a dose of about 0.0001 to about 10 mg/kg of patient body weight.

As noted above, the implantable device may be particularly suitable for treatment of ophthalmic diseases or improving one or more symptoms of the patient. Ophthalmic diseases can include diseases affecting one or more of the tissues or components of the eye, such as the sclera, iris, cornea, pupil, lens, ciliar body, conjunctiva, retina, optic nerve, macula, retinal blood vessels, vitreous body, and combinations thereof. In embodiments, the implantable device can be suitable for treating retinal disease, which includes any disease of the eye which is caused by or associated with the growth or proliferation of blood vessels or by blood vessel leakage. Non-limiting examples of retinal diseases that are treatable using the methods of the present disclosure include age-related macular degeneration (e.g., wet AMD, exudative AMD, etc.), retinal vein occlusion (RVO), central retinal vein occlusion (CRVO; e.g., macular edema following CRVO), branch retinal vein occlusion (BRVO), diabetic macular edema (DME), choroidal neovascularization (CNV; e.g., myopic CNV), iris neovascularization, neovascular glaucoma, post-surgical fibrosis in glaucoma, proliferative vitreoretinopathy (PVR), optic disc neovascularization, corneal neovascularization, retinal neovascularization, vitreal neovascularization, pannus, pterygium, vascular retinopathy, and diabetic retinopathies. Other ophthalmic diseases that can be treated with the implantable device of the present disclosure include amblyopia, astigmatism, cataracts, color blindness, dry eye, floaters, Bechet's Disease, Bietti's Crystalline Dystrophy, blepharitis, blepharospasm, cerebral visual impairment, Colomba, convergence insufficiency, corneal conditions, farsightedness, glaucoma, Graves' eye disease, idiopathic intracranial hypertension, low vision, macular edema, macular hole, macular pucker, myopia, ocular histoplasmosis syndrome, conjunctivitis, presbyopia, refractive errors, retinal detachment, retinitis pigmentosa, retinoblastoma, retinopathy of prematurity, Stargardt disease, usher syndrome, uvetis, vitreous detachment, and combinations thereof.

The manner in which the device of the present invention is implanted within the eye of a patient may vary as known to those skilled in the art. For example, the device may be inserted into the anterior segment (anterior chamber between the posterior surface of the cornea and the iris and/or the posterior chamber between the iris and front face of the vitreous humor), the posterior segment (e.g., anterior hyaloid membrane, vitreous humor, retina, choroid, etc.), or a combination thereof. When treating inflammatory eye conditions, such as uveitis, infection corneal ulcers, endophthalmitis, keratitis, conjunctivitis, and thyroid eye disease, it is generally desired that the therapeutic agent is delivered to the posterior segment. Further, the device may be inserted into the suprachoroidal space between the sclera and choroid to treat a retinal disease. When treating retinal diseases, such as glaucoma, age-related macular degeneration, and diabetic retinopathy, it is generally desired that the tyrosine kinase inhibitor is delivered to the posterior segment. To avoid having to pass through the blood-aqueous barrier, the implantable device may be directly inserted into the posterior segment, such as into the vitreous humor (“intravitreally”). Intravitreal injection techniques are well known in the art any may include, for instance, the use of a hollow needle through which the implant is passed. The needle may have a small diameter size (e.g., 18 to 30 gauge needle) so that the incision is self-sealing and the implantation occurs in a closed chamber. A self-sealing incision may also be formed using a conventional “tunneling” procedure in which a spatula-shaped scalpel is used to create a generally inverted V-shaped incision through the cornea. In a preferred mode, the instrument used to form the incision through the cornea remains in place (that is, extends through the corneal incision) during the procedure and is not removed until after implantation. Such incision-forming instrument either may be used to place the ocular implant or may cooperate with a delivery instrument to allow implantation through the same incision without withdrawing the incision-forming instrument. Of course, in other modes, various surgical instruments may be passed through one or more corneal incisions multiple times.

The implantable device can be placed in the desired location within the eye according to surgical procedures, such as those known in the art. For example, the implantable device can be inserted through a sclerotomy into the suprachoroid. In such a procedure, the sclera is cut to expose the suprachoroid. The implantable device can then be positioned and inserted on either side of the incision. Alternatively, a partial-thickness scleral trapdoor can be fashioned over the suprachoroid or an avascular region of the eye. The implantable device can then be inserted, and the scleral flap sewn back into place to secure the implant. Alternatively, the implantable device may be inserted so as to directly communicate with the vitreal chamber. To achieve this, a partial thickness scleral trap door flap is cut over an avascular region, such as the pars plana, to remove the eye coat. A hole (or holes) is made through the floor of the scleral bed to communicate with the base of the vitreous body through the pars plana. The implantable device is positioned over the hole within the scleral bed and the flap of the trap door is sewn back into place. Such placement of the implantable device will allow for the ready diffusion of the drug into the vitreous and into the intraocular structure.

The device may be implanted subcutaneously, orally, mucosally, etc., using standard techniques. The delivery route may be intrapulmonary, gastroenteral, subcutaneous, intramuscular, or for introduction into the central nervous system, intraperitoneum or for intraorgan delivery. Despite being particularly beneficial for treating and/or prohibiting retinal diseases, the implantable device may also be suitable for delivering a tyrosine kinase inhibitor to treat other conditions, such as cancer, allergies, inflammation, immunologically-mediated diseases, metabolic diseases, etc. In such embodiments, the device may be placed in a tissue site of a patient in, on, adjacent to, or near a tumor, such as a tumor of the pancreas, billary system, gallbladder, liver, small bowel, colon, brain, lung, eye, etc. If desired, the device may also be employed together with current systemic chemotherapy, external radiation, and/or surgery.

Regardless of the manner in which it is used, the implantable device may be sealed within a package (e.g., sterile blister package) prior to use. The materials and manner in which the package is sealed may vary as is known in the art. In one embodiment, for instance, the package may contain a substrate that includes any number of layers desired to achieve the desired level of protective properties, such as 1 or more, in some embodiments from 1 to 4 layers, and in some embodiments, from 1 to 3 layers. Typically, the substrate contains a polymer film, such as those formed from a polyolefin (e.g., ethylene copolymers, propylene copolymers, propylene homopolymers, etc.), polyester (e.g., polyethylene terephthalate, polyethylene naphthalate, polybutylene terephthalate, etc.), vinyl chloride polymer, vinyl chloridine polymer, ionomer, etc., as well as combinations thereof. One or multiple panels of the film may be sealed together (e.g., heat sealed), such as at the peripheral edges, to form a cavity within which the device may be stored. For example, a single film may be folded at one or more points and sealed along its periphery to define the cavity within with the device is located. To use the device, the package may be opened, such as by breaking the seal, and the device may then be removed and implanted into a patient.

IV. Method for forming the Device

Regardless of the particular components employed, the core and optional membrane layers may be formed through a variety of known techniques, such as by hot-melt extrusion, injection molding, solvent casting, dip coating, spray coating, microextrusion, coacervation, compression molding (e.g., vacuum compression molding), etc. In one embodiment, a hot-melt extrusion technique may be employed. Hot-melt extrusion is generally a solvent-free process in which the components of the core (e.g., hydrophobic polymer, therapeutic agent(s), optional excipients, etc.) may be melt blended and optionally shaped in a continuous manufacturing process to enable consistent output quality at high throughput rates. This technique is particularly well suited to various types of hydrophobic polymers, such as POE polymers.

During a hot-melt extrusion process, melt blending may occur at a temperature range of from about 40° C. to about 100° C., in some embodiments, from about 30° C. to about 80° C., in some embodiments from about 40° C. to about 65° C., and in some embodiments, in some embodiments from about 50° C. to about 65° C., to form a polymer composition. Any of a variety of melt blending techniques may generally be employed. For example, the components may be supplied separately or in combination to an extruder that includes at least one screw rotatably mounted and received within a barrel (e.g., cylindrical barrel). The extruder may be a single screw or twin screw extruder. For example, one embodiment of a single screw extruder may contain a housing or barrel and a screw rotatably driven on one end by a suitable drive (typically including a motor and gearbox). If desired, a twin-screw extruder may be employed that contains two separate screws. The configuration of the screw is not particularly critical, and it may contain any number and/or orientation of threads and channels as is known in the art. For example, the screw typically contains a thread that forms a generally helical channel radially extending around a core of the screw. A feed section and melt section may be defined along the length of the screw. The feed section is the input portion of the barrel where the olefin copolymer(s) and/or therapeutic agent(s) are added. The melt section is the phase change section in which the copolymer is changed from a solid to a liquid-like state. While there is no precisely defined delineation of these sections when the extruder is manufactured, it is well within the ordinary skill of those in this art to reliably identify the feed section and the melt section in which phase change from solid to liquid is occurring. Although not necessarily required, the extruder may also have a mixing section that is located adjacent to the output end of the barrel and downstream from the melting section. If desired, one or more distributive and/or dispersive mixing elements may be employed within the mixing and/or melting sections of the extruder. Suitable distributive mixers for single screw extruders may include, for instance, Saxon, Dulmage, Cavity Transfer mixers, etc. Likewise, suitable dispersive mixers may include Blister ring, Leroy/Maddock, CRD mixers, etc. As is well known in the art, the mixing may be further improved by using pins in the barrel that create a folding and reorientation of the polymer melt, such as those used in Buss Kneader extruders, Cavity Transfer mixers, and Vortex Intermeshing Pin mixers.

If desired, the ratio of the length (“L”) to diameter (“D”) of the screw may be selected to achieve an optimum balance between throughput and blending of the components. The L/D value may, for instance, range from about 10 to about 50, in some embodiments from about 15 to about 45, and in some embodiments from about 20 to about 40. The length of the screw may, for instance, range from about 0.05 to about 5 meters, in some embodiments from about 0.1 to about 4 meters, and in some embodiments, from about 0.5 to about 2 meters. The diameter of the screw may likewise be from about 5 to about 150 millimeters, in some embodiments from about 10 to about 120 millimeters, in some embodiments, from about 20 to about 80 millimeters, and in some embodiments from about 10 to about 20 millimeters. In addition to the length and diameter, other aspects of the extruder may also be selected to help achieve the desired degree of blending. For example, the speed of the screw may be selected to achieve the desired residence time, shear rate, melt processing temperature, etc. For example, the screw speed may range from about 10 to about 150 revolutions per minute (“rpm”), in some embodiments from about 20 to about 130 rpm, and in some embodiments, from about 10 to about 100 rpm. In embodiments, the screw speed has an RPM of from about 20 to about 70, such as from about 30 to about 65, such as from about 30 to about 40. The apparent shear rate during melt blending may also range from about 100 seconds⁻¹ to about 10,000 seconds⁻¹, in some embodiments from about 500 seconds⁻¹ to about 5000 seconds⁻¹, and in some embodiments, from about 800 seconds⁻¹ to about 1200 seconds⁻¹. The apparent shear rate is equal to 4Q/πR³, where Q is the volumetric flow rate (“m³/s”) of the polymer melt and R is the radius (“m”) of the capillary (e.g., extruder die) through which the melted polymer flows.

Once melt blended together, the resulting polymer composition may be in the form of pellets, sheets, fibers, filaments, etc., which may be shaped into the core using a variety of known shaping techniques, such as injection molding, compression molding, nanomolding, overmolding, blow molding, three-dimensional printing, etc. Injection molding may, for example, occur in two main phases—i.e., an injection phase and holding phase. During the injection phase, a mold cavity is filled with the molten polymer composition. The holding phase is initiated after completion of the injection phase in which the holding pressure is controlled to pack additional material into the cavity and compensate for volumetric shrinkage that occurs during cooling. After the shot has built, it can then be cooled. Once cooling is complete, the molding cycle is completed when the mold opens and the part is ejected, such as with the assistance of ejector pins within the mold. Any suitable injection molding equipment may generally be employed in the present disclosure. In one embodiment, an injection molding apparatus may be employed that includes a first mold base and a second mold base, which together define a mold cavity having the shape of the core. The molding apparatus includes a resin flow path that extends from an outer exterior surface of the first mold half through a sprue to a mold cavity. The polymer composition may be supplied to the resin flow path using a variety of techniques. For example, the composition may be supplied (e.g., in the form of pellets) to a feed hopper attached to an extruder barrel that contains a rotating screw (not shown). As the screw rotates, the pellets are moved forward and undergo pressure and friction, which generates heat to melt the pellets. A cooling mechanism may also be provided to solidify the resin into the desired shape of the core (e.g., disc, rod, etc.) within the mold cavity. For instance, the mold bases may include one or more cooling lines through which a cooling medium flows to impart the desired mold temperature to the surface of the mold bases for solidifying the molten material. The mold temperature (e.g., temperature of a surface of the mold) may range from about 30° C. to about 120° C., in some embodiments from about 60° C. to about 110° C., and in some embodiments, from about 30° C. to about 60° C.

Specifically, in embodiments, forming the implantable device includes melt-blending the core polymer matrix and one or more therapeutic agents in an extruder barrel at a first temperature and then mixing the core polymer matrix and therapeutic agent at a second temperature in the extruder barrel before extruding the mixture to form a core for the implantable device. The temperature during melt-blending can be the same or different from the temperature during mixing. Suitable temperatures range from about 40° C. to about 95° C., such as about 60° C. to about 80° C., such as from about 50° C. to about 70° C. Once extruded, the core can be cooled and then cut into suitable shapes to form the implantable device.

Specifically, in embodiments, forming the implantable device includes melt-blending the core polymer matrix and one or more therapeutic agents in an extruder barrel at a first temperature and then mixing the core polymer matrix and therapeutic agent at a second temperature in the extruder barrel before extruding the mixture to form a core for the implantable device. Additional, melting and/or blending steps can be utilized to further process the core polymer matrix and one or more therapeutic agents. Generally, however, melting and blending of the membrane polymer matrix takes place at temperatures less than 80° C., such as between about 45° C. and 70° C. The temperature during melt-blending can be the same or different from the temperature during mixing. Suitable temperatures range from about 45° C. to about 75° C., such as about 50° C. to about 65° C. Once extruded, the core can be cooled and then cut into suitable shapes to form the implantable device.

Further, a membrane layer can be added to the core via melt-extrusion, dip-coating, dye-casting, etc. For instance, in embodiments a membrane polymer matrix including one or more hydrophobic polymers is melt-blended in an extruder barrel at a first temperature. Suitable temperatures can range from about 40° C. to about 80° C., such as from about 50° C. to about 70° C. Additional, melting and/or blending steps can be utilized to further process the membrane polymer matrix. Generally, however, melting and blending of the membrane polymer matrix takes place at temperatures less than 80° C., such as between about 50° C. and 70° C. Optionally, the membrane polymer matrix can include one or more therapeutic agents as described hereinabove. The method includes extruding the membrane polymer matrix on an outer surface of the core, cooling the membrane polymer matrix and the core polymer matrix to form an implantable device having a core and membrane structure. In embodiments, both the core polymer matrix and membrane polymer matrix can be co-extruded.

In other embodiments, the membrane can be added to the core via other techniques that do not include melt-processing. For instance, the core can be dipped into a membrane polymer composition including a POE polymer dispersed in a suitable solvent (e.g., organic solvent). The POE polymer can then coat the core providing a membrane thereon. The core can be dipped into the POE polymer composition as desired to build up the membrane to the desired thickness.

As indicated above, another suitable technique for forming an implantable device of the desired shape and size is three-dimensional printing. During this process, the polymer composition may be incorporated into a printer cartridge that is readily adapted for use with a printer system. The printer cartridge may, for example, contain a spool or other similar device that carries the polymer composition. When supplied in the form of filaments, for example, the spool may have a generally cylindrical rim about which the filaments are wound. The spool may likewise define a bore or spindle that allows it to be readily mounted to the printer during use. Any of a variety of three-dimensional printer systems can be employed in the present disclosure. Particularly suitable printer systems are extrusion-based systems, which are often referred to as “fused deposition modeling” systems. For example, the polymer composition may be supplied to a build chamber of a print head that contains a platen and gantry. The platen may move along a vertical z-axis based on signals provided from a computer-operated controller. The gantry is a guide rail system that may be configured to move the print head in a horizontal x-y plane within the build chamber based on signals provided from controller. The print head is supported by the gantry and is configured for printing the build structure on the platen in a layer-by-layer manner, based on signals provided from the controller. For example, the print head may be a dual-tip extrusion head.

Compression molding (e.g., vacuum compression molding) may also be employed. In such a method, a layer of the device may be formed by heating and compressing the polymer compression into the desired shape while under vacuum. More particularly, the process may include forming the polymer composition into a precursor that fits within a chamber of a compression mold, heating the precursor, and compression molding the precursor into the desired layer while the precursor is heated. The polymer composition may be formed into a precursor through various techniques, such as by dry power mixing, extrusion, etc. The temperature during compression may range from about 50° C. to about 120° C., in some embodiments from about 60° C. to about 110° C., and in some embodiments, from about 70° C. to about 90° C. A vacuum source may also apply a negative pressure to the precursor during molding to help ensure that it retains a precise shape. Examples of such compression molding techniques are described, for instance, in U.S. Pat. No. 10,625,444 to Treffer, et al., which is incorporated herein in its entirety by reference thereto.

### EXAMPLES

### Test Methods

Drug Release: The release of a therapeutic agent from a polymeric implant may be determined using an in vitro method. More particularly, implantable device samples may be placed in 5 milliliters of an aqueous PBS buffer solution. The solutions are enclosed in centrifuge tubes. The tubes are then placed into a temperature-controlled incubator and continuously shaken at 100 rpm. A temperature of 37° C. is maintained through the release experiments to mimic in vivo conditions. Samples are taken in regular time intervals by completely exchanging the buffer solution. The concentration of a therapeutic agent in solution may be determined via an HPLC method which is described below. From these data, the amount of the therapeutic agent released per sampling interval (milligram per day) may be calculated and plotted over time (days). Further, the cumulative release ratio of the therapeutic agent may be calculated as a percentage by dividing the amount of the therapeutic agent released at each sampling interval by the total amount of therapeutic agent initially present, and then multiplying this number by 100. This percentage is then plotted over time (days).

### Example 1

A rod-shaped monolithic implant containing an IgG antibody having a molecular weight around 150 kDa was produced via extrusion. The device contained 55 wt. % POE polymer and 45 wt. % IgG antibody. The POE polymer contained DETOSU as acetal monomers and HDO as polyol monomers. The POE polymer did not contain any aliphatic ester modified polyol monomers. The device was formed by melt extruding the components using a 11 mm twin-screw extruder. Extrusion was accomplished using a screw speed of 50 rpm with barrel temperatures set to achieve a nominal melt temperature of 63° C.

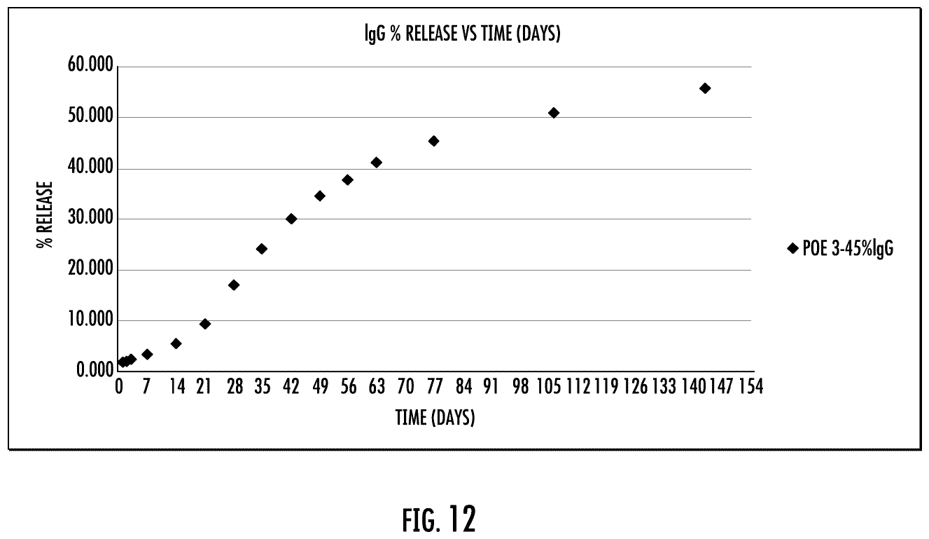

The extruded rod had a diameter of approximately 2 mm and was cut to a length of 1 cm for elution testing. The release of IgG from the rods was measured in PBS buffer in a shaking incubator maintained at 37° C. At regular intervals, the buffer was exchanged with fresh buffer, and the removed buffer characterized using the previously described HPLC method to measure the concentration of IgG released. The resulting cumulative release rate (%) is shown in FIG. 12. (Results shown in Table 1 below).

TABLE 1
Percent drug release from 45% loaded core (Example 1)

| | Average |
|---|---|
| Time | (%) |
| (Days) | Release |
| 1 | 1.75 |
| 7 | 3.37 |
| 14 | 5.47 |
| 21 | 9.53 |
| 28 | 16.91 |
| 35 | 24.06 |
| 42 | 29.90 |
| 49 | 34.44 |
| 56 | 37.70 |
| 63 | 41.06 |
| 77 | 45.36 |
| 106 | 50.87 |
| 143 | 55.57 |

### Example 2

A rod-shaped monolithic implant containing POE polymer (HDO) having a Tg of 15° C. and trastuzumab was produced via extrusion. The device contained 55 wt. % POE polymer and 45 wt. % POE polymer. The POE polymer was first cryogrinded to make into powder form. Then, the cryogrinded POE polymer was blended with the trastuzumab. The blend was fed slowly and compounded with via 11 mm twin-screw extruder. Compounded filaments were produced, and the diameters of the compounded filaments were 3 mm. The processing temperature in the twin-screw extruder was less than 70° C. The filaments were cut into 1 cm long piece to perform in vitro release study of trastuzumab.

Rods with dimensions 3 mm (D)×1 cm (L) were used for elution testing. The release of trastuzumab from rods into PBS buffer was measured in a shaking incubator maintained at 37° C. At regular intervals, the buffer was exchanged with fresh buffer, and the removed buffer characterized using HPLC-UV analysis method. The SEC stability analysis was done to understand any aggregation or fragmentation of the Trastuzumab after extrusion or during elution.

The resulting cumulative release rate (%) is shown in FIG. 9 and the Cumulative release/surface area versus time is shown in FIG. 10. (Results shown in Tables 2-3 below).

TABLE 2

| | % Release (Cumulative) | | | | | | | |
|---|---|---|---|---|---|---|---|---|
| | 1 | 2 | 3 | 7 | 10 | 14 | 17 | 21 |
| 55% POE-HDO 100/45% | 1.99 | 2.10 | 2.14 | 2.34 | 2.59 | 2.87 | 3.09 | 3.3 |
| Trastuzumab | | | | | | | | |

TABLE 3

| | Cumulative release (mg)/surface | | | | | | | |
|---|---|---|---|---|---|---|---|---|
| | area (cm\hat{ }2) vs Time (Days) | | | | | | | |
| | 1 | 2 | 3 | 7 | 10 | 14 | 17 | 21 |
| 55% POE-HDO 100/45% | 0.35 | 0.36 | 0.37 | 0.41 | 0.45 | 0.50 | 0.54 | 0.6 |
| Trastuzumab | | | | | | | | |

Table 4 illustrates the release per day for Example 2.

TABLE 4

| | Release Per Day (mg) | | | | | | | |
|---|---|---|---|---|---|---|---|---|
| | 1 | 2 | 3 | 7 | 10 | 14 | 17 | 21 |
| 55% POE-HDO 100/ | 0.38 | 0.02 | 0.01 | 0.04 | 0.05 | 0.05 | 0.04 | 0.05 |
| 45% Trastuzumab | | | | | | | | |

To verify that trastuzumab was not altered during implant manufacture, the HPLC chromatograms were taken before and after extrusion. FIG. 11 illustrates HPLC chromatograms taken before extrusion for Example 2 and FIG. 12 illustrates HPLC chromatograms taken after extrusion for Example 2.

### Examples 3-4

Two different POE polymers were assessed, one having POE polymer containing HDO with 1 wt % GL (Example 3) and one having POE polymer containing HDO with 3 wt % GL. To each POE polymer IgG was loaded to a weight percentage of 45%. The implants were then subjected to hot melt extrusion and extruded to form a filament. The implants were made into cylindrical shapes measuring 3.5 mm in diameter and 1 cm in length. Implants were weighed and placed into Eppendorf tubes with buffer solution (pH=7.4). The tubes were placed into incubators at 37° C. and 100 rpm, and periodically sampled over the course of six months. HPLC-UV was performed to quantify IgG release. Light microscopy techniques were utilized to understand the degradation behavior of the polymers.

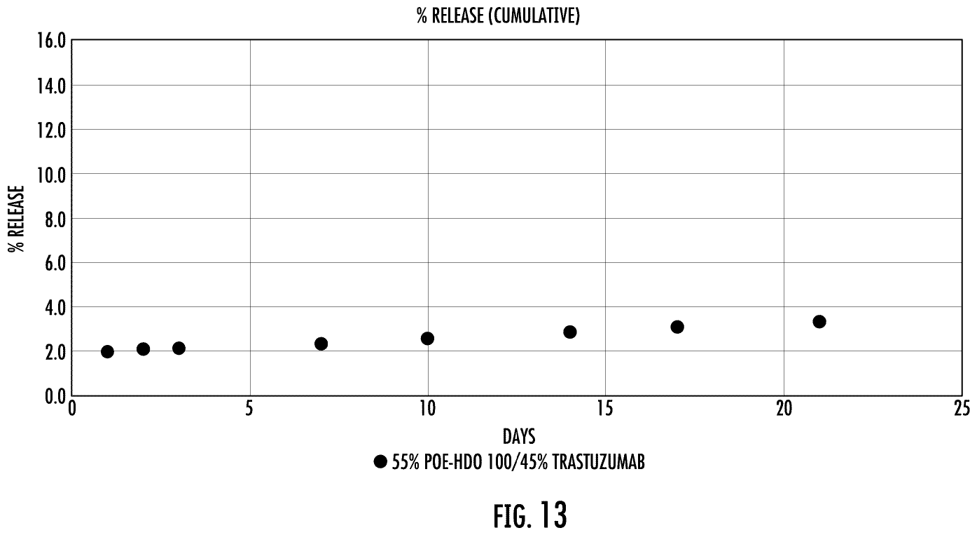

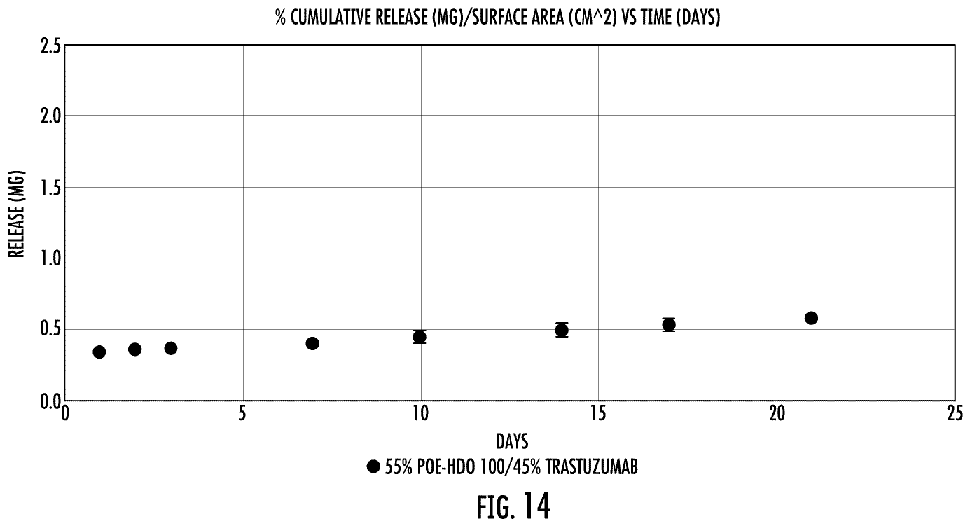

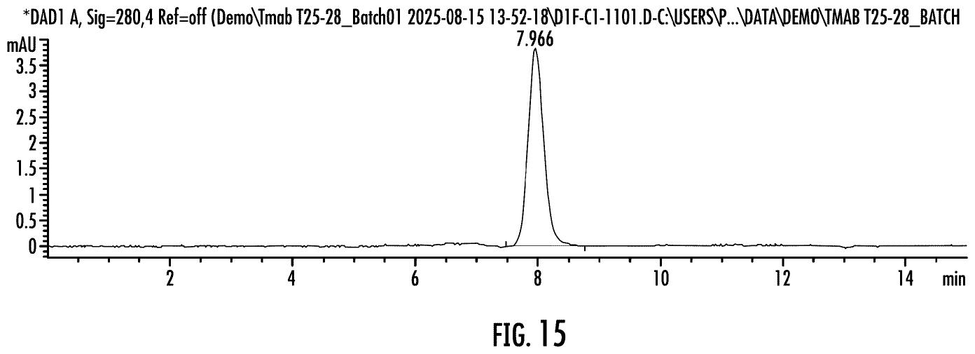

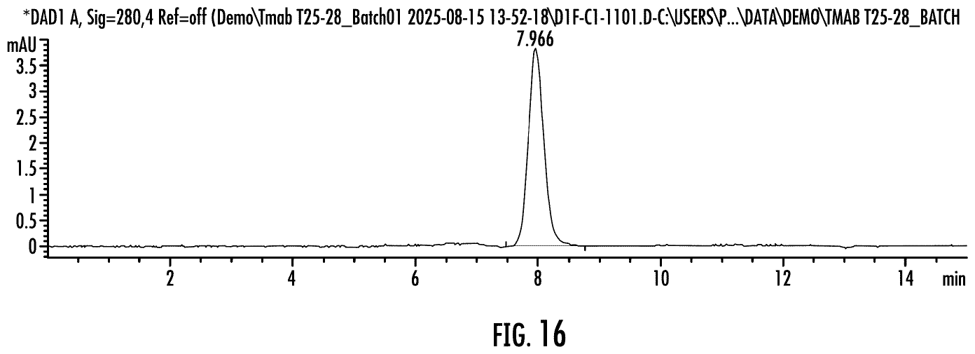

Light microscopy images showing the degradation behavior of the implants for POE polymer containing 1 wt. % GL are shown in FIG. 13 and the POE polymer containing 3 wt. % GL are shown in FIG. 14. The cumulative release for each Example is shown in FIG. 15 and the cumulative release per surface are versus time is shown in FIG. 16. (Results shown in Tables 5-6 below).

TABLE 5
% Release (Cumulative)

| | 1% HDO-GL/45% IgG | 3% HDO-GL/45% IgG |
|---|---|---|
| Days | (Example 3) | (Example 4) |
| 1 | 0.9 | 1.1 |
| 2 | 1.1 | 1.5 |
| 3 | 1.4 | 1.9 |
| 7 | 2.7 | 3.3 |
| 14 | 7.4 | 9.5 |
| 21 | 15.3 | 19.6 |
| 28 | 24.7 | 30.3 |
| 35 | 29.7 | 35.1 |
| 42 | 32.7 | 37.7 |
| 56 | 35.5 | 40.3 |
| 70 | 37.4 | 42.1 |
| 84 | 38.5 | 43.1 |
| 98 | 40.2 | 44.8 |
| 112 | 41.5 | 46.1 |
| 126 | 42.3 | 46.9 |
| 140 | 43.0 | 47.7 |
| 154 | 43.8 | 48.7 |
| 168 | 45.0 | 50.8 |
| 182 | 47.9 | 54.8 |
| 189 | 55.0 | 61.0 |
| 203 | 55.5 | 61.8 |

TABLE 6
Cumulative release (mg)/surface area (cm\hat{ }2) vs Time (Days)

| | 1% HDO-GL/45% IgG | 3% HDO-GL/45% IgG |
|---|---|---|
| Days | (Example 3) | (Example 4) |
| 1 | 0.3 | 0.4 |
| 2 | 0.4 | 0.5 |
| 3 | 0.5 | 0.7 |
| 7 | 0.9 | 1.2 |
| 14 | 2.6 | 3.5 |
| 21 | 5.4 | 7.2 |
| 28 | 8.8 | 11.1 |
| 35 | 10.5 | 12.9 |
| 42 | 11.6 | 13.8 |
| 56 | 12.6 | 14.8 |
| 70 | 13.2 | 15.5 |
| 84 | 12.9 | 15.8 |
| 98 | 13.5 | 16.4 |
| 112 | 14.0 | 16.9 |
| 126 | 14.3 | 17.2 |
| 140 | 14.5 | 17.5 |
| 154 | 14.8 | 17.9 |
| 168 | 15.2 | 18.6 |
| 182 | 16.2 | 20.1 |
| 189 | 18.8 | 22.4 |
| 203 | 18.9 | 22.7 |

### Examples 5-6

Two different POE polymers were assessed, one having POE polymer containing HDO with 1 wt % GL (Example 5) and one having POE polymer containing HDO with 3 wt % GL (Example 6). To each POE polymer axinitib was loaded to a weight percentage of 45%. The implants were then subjected to hot melt extrusion and extruded to form a filament. The implants were made into cylindrical shapes measuring 3.5 mm in diameter and 1 cm in length. Implants were weighed and placed into Eppendorf tubes with buffer solution (pH=7.4). The tubes were placed into incubators at 37° C. and 100 rpm, and periodically sampled over the course of six months. HPLC-UV was performed to quantify IgG release. Light microscopy techniques were utilized to understand the degradation behavior of the polymers.

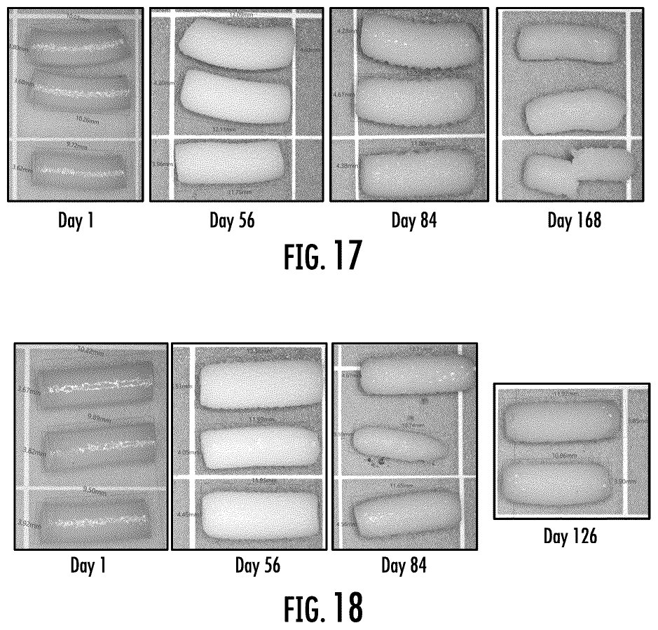

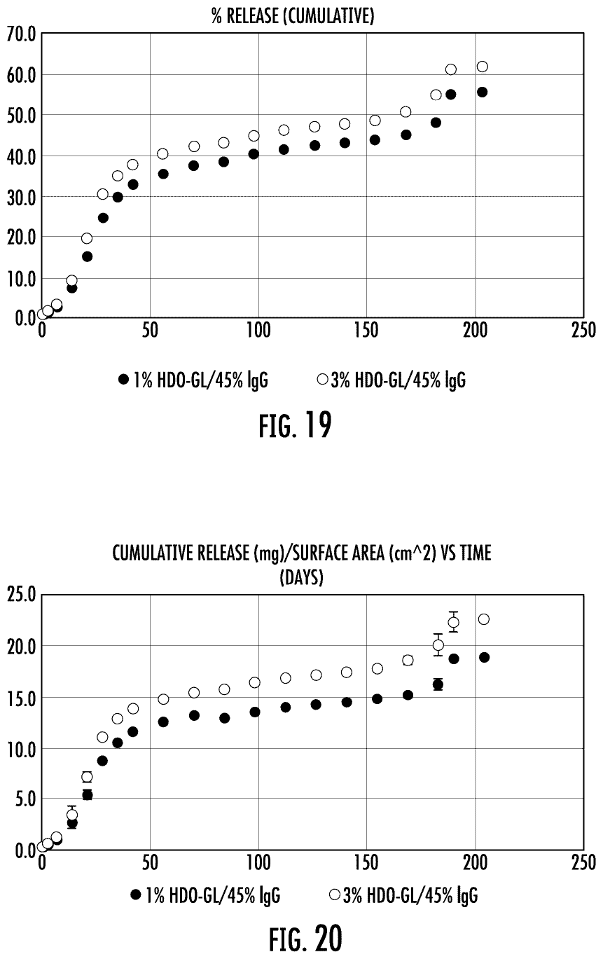

Light microscopy images showing the degradation behavior of the implants for POE polymer containing 1 wt. % GL are shown in FIG. 17 and the POE polymer containing 3 wt. % GL are shown in FIG. 18. The cumulative release for each Example is shown in FIG. 19 and release per surface area over time is shown in FIG. 20. (Results shown in Table 7-8 below).

TABLE 7
Total Cumulative Release (ug)

| | 1% HDO-GL/45% Axitinib | 3% HDO-GL/45% Axitinib |
|---|---|---|
| Days | (Example 5) | (Example 6) |
| 1 | 0.93 | 0.86 |
| 2 | 1.73 | 1.67 |
| 3 | 2.47 | 2.54 |
| 7 | 3.61 | 3.89 |
| 14 | 4.8 | 5.2 |
| 21 | 6.0 | 6.6 |
| 28 | 7.5 | 9.9 |
| 34 | 8.9 | 12.6 |
| 42 | 20.8 | 20.6 |
| 56 | 23.3 | 24.3 |
| 70 | 32.4 | 46.3 |
| 84 | 46.7 | 58.9 |
| 98 | 56.9 | 69.6 |
| 112 | 65.9 | 78.6 |
| 126 | 75.8 | 90.2 |
| 140 | 75.9 | 90.2 |
| 156 | 76.3 | 90.6 |
| 168 | 76.7 | 94.0 |
| 189 | 76.8 | 94.1 |
| 197 | 76.9 | 94.2 |

TABLE 8
Cumulative release (ug)/surface area (cm\hat{ }2) vs Time (Days)

| | 1% HDO-GL/45% | 3% HDO-GL/45% |
|---|---|---|
| | Axitinib | Axitinib |
| Days | (Example 5) | (Example 6) |
| 1 | 0.85 | 0.78 |
| 2 | 1.57 | 1.51 |
| 3 | 2.23 | 2.30 |
| 7 | 3.28 | 3.51 |
| 14 | 4.3 | 4.7 |
| 21 | 5.4 | 6.0 |
| 28 | 6.8 | 9.0 |
| 34 | 8.1 | 11.3 |
| 42 | 18.5 | 18.3 |
| 56 | 20.7 | 21.6 |
| 70 | 28.9 | 42.4 |
| 84 | 41.5 | 53.6 |
| 98 | 50.6 | 63.2 |
| 112 | 58.8 | 71.3 |
| 126 | 67.7 | 81.7 |
| 140 | 67.8 | 81.7 |
| 156 | 68.1 | 82.1 |
| 168 | 68.5 | 85.2 |
| 189 | 68.6 | 85.3 |
| 197 | 68.7 | 85.4 |

### Example 7

Example 7 is an illustrative examples of melt-blending two POE polymers having different Tgs to arrive at a POE polymer having a desired Tg within a specific range suitable for melt processing (e.g., melt extrusion) without degrading the therapeutic agent during processing.

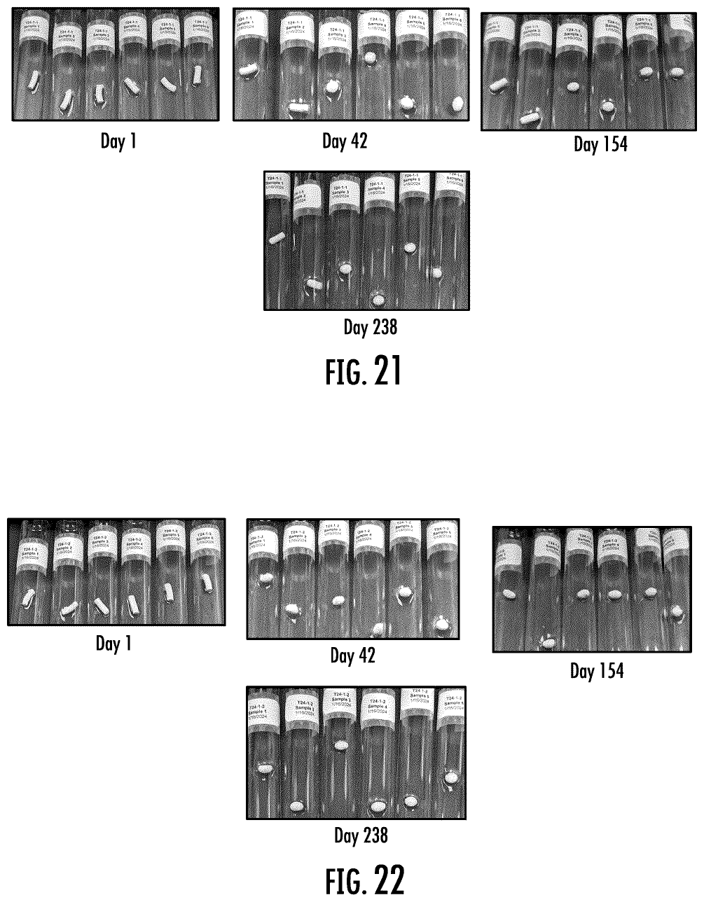

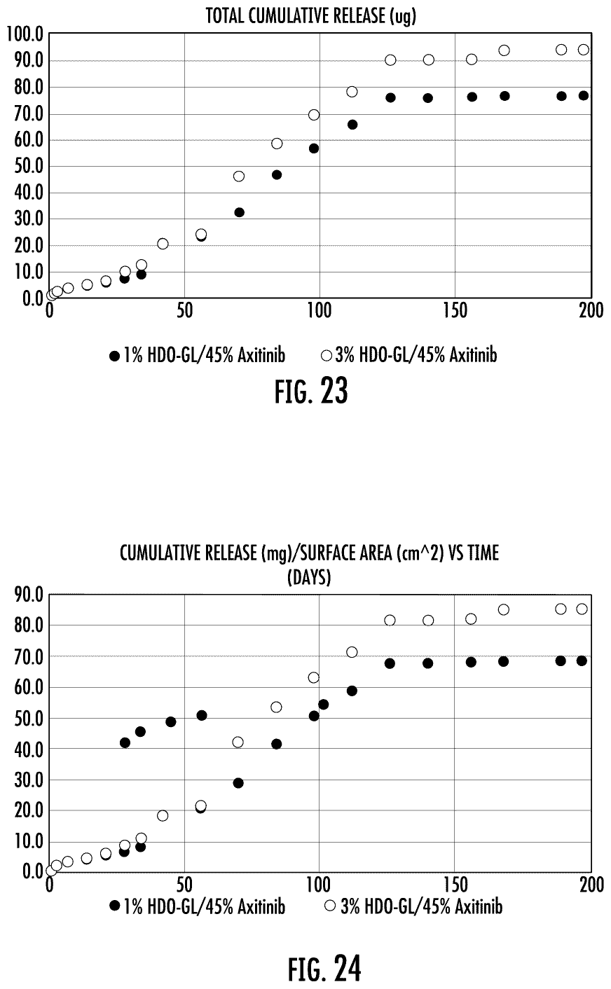

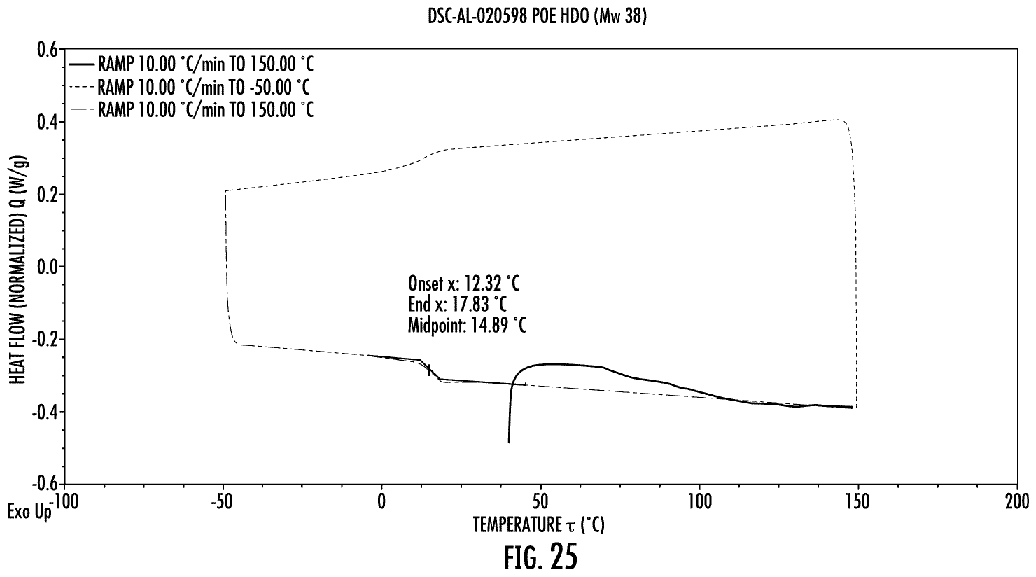

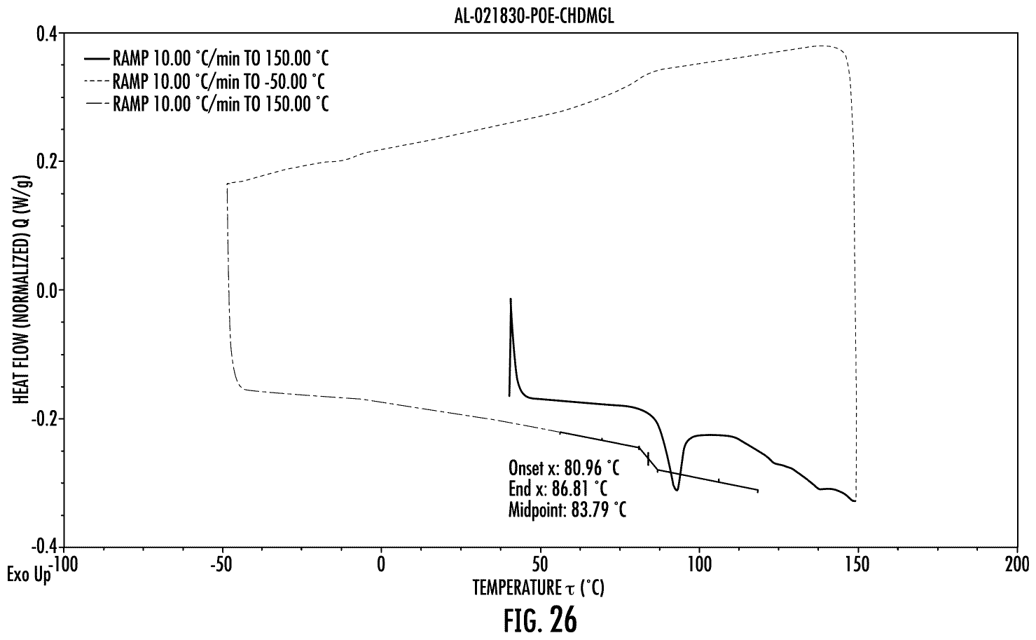

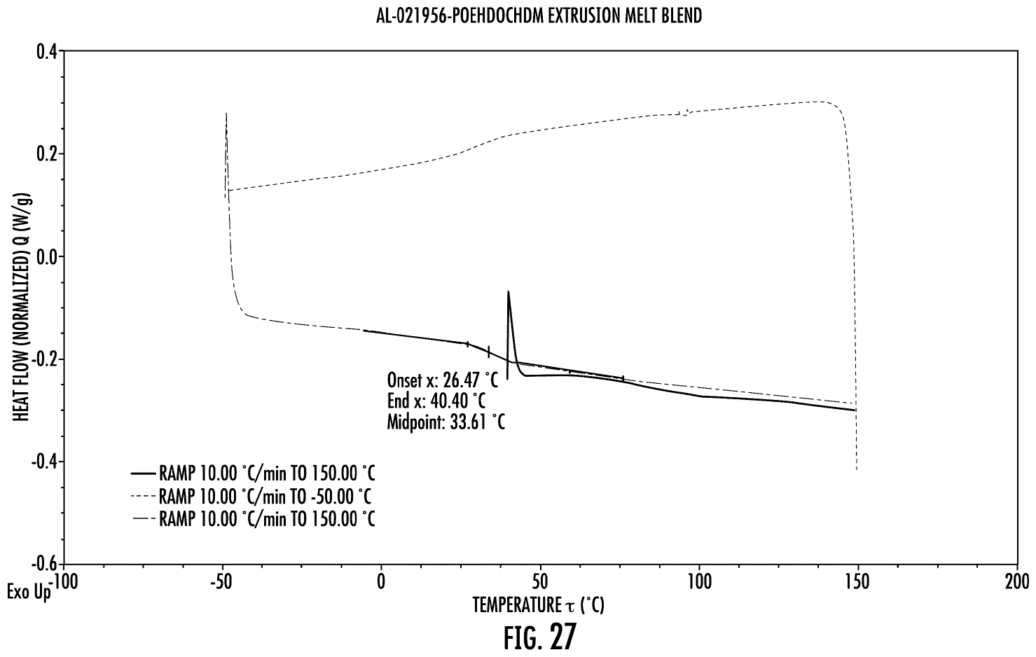

A first POE polymer (HDO) had a Tg around 18° C. and a second POE polymer (CHDM-GL) had a Tg around 89° C. As noted above, the Tg of the POE polymer system is important for melt blending and melt extrusion processing. For each of the single polymers, the processing temperature needed for extrusion is around 60° C. for the first POE polymer (HDO) and around 130° C. for the second polymer (CHDM-GL). As noted, at processing temperatures above 80° C., many therapeutic agents, especially large molecular weight compounds (e.g., over 0.5 kDa) often degrade during processing, rendering them ineffective for use. To bring down the processing temperature of the second POE polymer (CHDM-GL), the first POE polymer was melt blended with the second POE polymer in a 50:50 by wt. % ratio. The melt blending process was completed at a processing temperature of about 95° C. FIGS. 21 and 22 show the DSC of the first POE polymer and second POE polymer prior to melt blending. FIG. 23 shows the DSC of the melt blended polymers. The DSC after the melt blending shows that the resulting POE polymer blend has a Tg around 34° C. This POE polymer blend is suitable for melt extrusion processing where the processing temperature is about 70° C.

### Definitions

As used herein, “glass transition temperature” or Tg, refers to the temperature at which an amorphous or partly amorphous material (in the case of polymers) undergoes a phase change from a hard, solid amorphous or partly amorphous material to a soft, rubbery liquid. The glass transition temperature of a POE composition can readily be determined by differential scanning calorimetry (DSC).

As used herein, ranges and amounts can be expressed as “about” a particular value or range. “About” is intended to also include the exact amount. Hence “about 5 percent” means “about 5 percent” and also “5 percent.” “About” means within typical experimental error for the application or purpose intended.

As used herein, “optional” or “optionally” means that the subsequently described event or circumstance does or does not occur, and that the description includes instances where said event or circumstance occurs and instances where it does not. For example, an optional component in a method, composition, or device means that the component may be present or may not be present in the method, composition, or device.

As used herein, the term “substantially free” means no more than an insignificant trace amount present and encompasses completely free (e.g., 0 molar % up to 0.01 molar %).

The methods, compositions, and devices of the present disclosure, including components thereof, can comprise, consist of, or consist essentially of the essential elements and limitations of the disclosure described herein.

These and other modifications and variations of the present invention may be practiced by those of ordinary skill in the art, without departing from the spirit and scope of the present invention. In addition, it should be understood that aspects of the various embodiments may be interchanged both in whole or in part. Furthermore, those of ordinary skill in the art will appreciate that the foregoing description is by way of example only and is not intended to limit the invention so further described in such appended claims.

What is claimed is:

1. An implantable device for ocular insertion, the implantable device comprising: a core comprising a core polymer matrix within which is dispersed one or more therapeutic agents, wherein core polymer matrix comprises a hydrophobic polymer comprising a first poly ortho ester (POE) polymer, wherein the POE polymer has a Tg of between about −20° C. and 40° C. as determined in accordance with ASTM E1640-18.

2. The implantable device of claim 1, wherein the one or more therapeutic agents constitute from about 40 wt. % to about 70 wt. % of the core.

3. The implantable device of claim 1, wherein the POE polymer has an acetal monomer content of from about 20 mol. % to about 80 mol. %.

4. The implantable device of claim 1, wherein the POE polymer has a polyol monomer content of from about 20 mol. % to about 80 mol. %.

5. The implantable device of claim 4, wherein the polyol monomer content comprises from about 1 mol. % to about 60 mol. % of one or more modified polyols.

6. The implantable device of claim 1, wherein the POE polymer has a molecular weight of from about 1 kDa to about 100 kDa.

7. The implantable device of claim 1, wherein the first POE polymer has a T_{g} of between about 0° C. and 40° C. as determined in accordance with ASTM E1640-18.

8. The implantable device of claim 1, wherein the one or more therapeutic agents comprise a macromolecular compound having a molecular weight of about 1 kDa or more.

9. The implantable device of claim 1, wherein the one or more therapeutic agents comprise a protein, peptide, enzyme, antibody, interferon, interleukin, blood factor, vaccine, nucleotide, lipid, or an analogue, derivative, or combination thereof.

10. The implantable device of claim 1, wherein the one or more therapeutic agents include a tyrosine kinase inhibitor.

11. The implantable device of claim 1, wherein the one or more therapeutic agents include a VEGF antagonist.

12. The implantable device of claim 1, wherein the one or more therapeutic agents include a steroidal agent.

13. The implantable device of claim 1, wherein the device is in the form of a cylinder having a length of from about 1 millimeter to about 250 millimeters and cross-sectional diameter of from about 0.1 millimeters to about 10 millimeters.

14. The implantable device of claim 1, wherein the device is in the form of a punctal plug.

15. The implantable device of claim 1, wherein the core is formed from a hot melt extrusion process.

16. A method for prohibiting and/or treating an ophthalmic disease in a patient in need thereof, the method comprising inserting the device of claim 1 in an eye of a patient.

17. The method of claim 16, wherein the device is inserted into an anterior segment of the eye.

18. The method of claim 16, wherein the device is inserted into a posterior segment of the eye.

19. The method of claim 16, wherein the device is inserted into a vitreous humor of the eye.

20. The method of claim 16, wherein the device is inserted into a suprachoroidal space of the eye.
